(19) United States
(12) Patent Application Publication
(10) Pub. No.: US 2010/0226990 A1
(43) Pub. Date: Sep. 9, 2010

(54) Method of Producing Porous Microparticles

(75) Inventors: Anne Marie Healy, Dublin (IE); Bernard Mcdonald, Monaghan (IE); Owen I. Corrigan, Dublin (IE); Lidia Tajber, Dublin (IE)

Correspondence Address: JACOBSON HOLMAN PLLC, 400 SEVENTH STREET N.W., SUITE 600, WASHINGTON, DC, 20004, US

(73) Assignee: THE PROVOST, FELLOWS AND SCHOLARS OF THE COLLEGE O, Dublin (IE)

(21) Appl. No.: 12/223,282

(22) PCT Filed: Jan. 29, 2007
(86) PCT No.: PCT/IE2007/000006

§ 371 Date: Jul. 25, 2008

(30) Foreign Application Priority Data
Jan. 27, 2006 (IE) 2006/0052

(51) Int. Cl. A61K 9/14; A61K 38/00; A61K 38/16; A61K 31/58; A61K 31/549; A61K 31/505; A61K 31/353; A61K 31/573; A61K 31/606; A61K 38/47; A61K 38/48; A61P 3/00; A61P 37/00; A61P 31/00; B29B 9/00

(52) U.S. Cl. 424/489; 514/2; 514/12; 514/174; 514/223.2; 514/272; 514/456; 514/179; 514/166; 424/94.61; 424/94.64; 264/13; 977/773

(57) ABSTRACT

A method of preparing porous microparticles comprises the steps of combining one or more organic compounds with a volatile system, and drying the system thus formed to provide substantially pure porous microparticles of the organic compound or composite porous microparticles of combinations of organic compounds. Organic compounds used in the method may be one or more of a bioactive, a pharmaceutically acceptable excipient, a pharmaceutically acceptable adjuvant or combinations thereof. The invention also relates to porous microparticles produced by such a method, and pharmaceutical compositions comprising such porous microparticles.

### INTRODUCTION

The invention relates to a method of producing porous microparticles and porous microparticles produced by such a method.

The use of mixed solvent systems in spray drying to produce microparticles of organic pharmaceuticals has previously been described.

Examples of prior art that disclose the spray drying of bioactive pharmaceuticals from mixed solvent systems are listed below:

Matsuda et al., (J. Pharm. Pharmacol. 44, 627-633 (1992)) spray dried frusemide from a chloroform/methanol (4:1) solvent mixture;

Corrigan et al. (Drug Devel. Ind. Pharm. 9, 1-20 (1983); Int. J. Pharm. 18, 195-200 (1984)) spray dried a number of thiazide compounds from ethanol and ethanol water mixtures;

Gilani et al. (J. Pharm. Sci. 94(5), 1048-1059 (2005)) spray dried cromolyn sodium (CS) under constant operation conditions from different water to ethanol feed ratios (50:50-0:100). CS particles spray dried from absolute ethanol were described as being of uniform elongated shape whereas the other samples were described as consisting mainly of particles with irregular shape;

Corrigan et al. (Int. J. Pharm. 273(1-2), 171-82 (2004)) spray dried salbutamol sulphate from an ethanol/water (75:25) solvent mixture; and

Corrigan et al. (Int. J. Pharm., 262(1-2) (2003)), spray dried bendroflumethiazide from an ethanol/water (95:5) solvent mixture.

However none of these systems produce porous microparticles.

Composite microparticles have been produced for example by spray drying a mixed solution of salbutamol sulphate and ipratropium bromide from ethanol/water. The ethanol:water was present in one of the following ratios: 84:16, 85:15 and 89:11 v/v (Corrigan et al., Int. J. Pharm. 322(1-2), 22-30 (2006)). Ozeki et al. (J. Control. Release 107(3), 387-395 (2005)) used a novel 4-fluid nozzle spray dryer to also prepare composite microparticles of a water-insoluble drug, flurbiprofen (FP), and a water-soluble drug, sodium salicylate (SS). An ethanol solution of FP and an aqueous SS solution were simultaneously introduced through different liquid passages in the 4-fluid nozzle spray dryer and then spray-dried. Again, none of the particles produced by these systems were porous.

Thus there is a wealth of prior art relating to a process of forming microparticles that teaches forming microparticles by the process of spray drying results in microparticles with a solid or intact (non-porous) wall.

Porous particles for delivery to the respiratory tract are described in U.S. Pat. No. 6,309,623 and U.S. Pat. No. 6,433,040. U.S. Pat. No. 6,565,885 describes spray drying for forming powder compositions of this type. Larger porous particles are also described in U.S. Pat. No. 6,447,753 and Edwards et al., Large porous particles for pulmonary drug delivery, Science, 276, 1868-1871 (1997). The prior art describes the production of hollow porous particles by spray drying an emulsion consisting of a bioactive agent, a surfactant and a blowing agent. The blowing agent is typically a volatile liquefied gas such as HFA propellant, or a volatile liquid such as carbon tetrachloride. A surfactant is required to stabilise the emulsion and remains as a residual/contaminant in the particles.

Zhou et al. (J. Materials Sci., 36, 3759-3768 (2001)) describe the production of porous polymer (polymethyl methacrylate, PMMA) microparticles by spray drying solutions of the polymers dissolved in mixed solvent systems. PMMA is a biostable polymer, practically insoluble in water and its medical applications include the production of bone cement and hard contact lenses. The production of porous particles of inorganic materials, produced by a similar process, is also described by Leong (J. Aerosol Sci., 12, 417-435 (1981) and J. Aerosol Sci., 18, 525-552 (1987)).

Polymeric nanoparticles of polymer (Eudragit L100) and polymer-drug (ketoprofen) composites have also been prepared by a spray drying process as described by Raula et a. (Int. J. Pharm., 284, 13-21 (2004)). These nanoparticles had geometric mean diameters less than 150 nm and maximum diameters (from SEM scans) of less than 500 nm. Some of the particles prepared were described as having shrivelled and brainlike structures while others were described as having blistery surfaces or popcorn-structures. The inclusion of drug did not influence the particle formation and ketoprofen content was only 10% w/w. The authors of the study concluded that the polymer controls the particle formation process.

Corrigan et al. have prepared cauliflower-like particles of spray dried polyethylene glycol polymer from a water/ethanol solution (Int. J. Pharm., 235, 193-205 (2002)) and brainlike particles of spray dried chitosan polymer and chitosan-salbutamol composites with corrugated surfaces, spray dried from acetic acid solution (Eur. J. Pharm. Biopharm., 62, 295-305 (2006)).

U.S. Pat. No. 4,610,875 (Panoz and Corrigan) describes the production of amorphous forms of drugs with high solubility, by a spray drying process. The amorphous form of the drug was stabilised by the presence of polyvinylpyrrolidone (PVP) as a stabilizer and an agent inhibiting crystallisation. The drug or drug-PVP combination was spray dried from water or from a water/alcohol mix.

### STATEMENTS OF INVENTION

According to the invention there is provided a method of preparing porous microparticles comprising the steps of:
combining one or more organic compounds with a volatile solvent system; and
drying the system thus formed to provide substantially pure porous microparticles of the organic compound or composite porous microparticles of combinations of organic compounds.

Preferably the organic compound may be one or more of a bioactive, a pharmaceutically acceptable excipient, a pharmaceutically acceptable adjuvant, or combinations thereof.

The method of the present invention provides an efficient method of manufacturing porous microparticles. In particular the method of the present invention may be considered as a simplified method of producing porous microparticles of organic compounds. For example the method in accordance with the present invention does not require the presence of a surfactant and no emulsion is formed prior to drying (unlike the known systems described for example in U.S. Pat. No. 6,447,753). In the method of the invention an organic compound is dissolved in a volatile solvent solution, and upon drying the volatile solvent solution (system) evaporates thereby providing substantially pure porous microparticles.

In accordance with the invention, the term substantially pure can be understood to mean consisting of only that material (for example only bioactive or only pharmaceutically acceptable excipient or only pharmaceutically acceptable adjuvant or combinations thereof) or composite (for example bioactive and pharmaceutically acceptable excipient and/or pharmaceutically acceptable adjuvant or a mixture of bioactives; a mixture of pharmaceutically acceptable excipients or a mixture of pharmaceutically acceptable adjuvants or combinations thereof) with none or only trace amounts (typically less than 1%) of any other component present.

The substantially porous microparticles that are produced in accordance with the present invention may be particularly suited for use for example in drug delivery such as drug delivery by respiratory methods (inhalation and the like). The microparticles produced by the method of the invention may be nanoporous. This may render the microparticles particularly suitable for drug delivery systems as the pores may increase the total surface area of the microparticles. Additionally, the pores of the microparticles may provide one or more of the following advantageous features:
reduce the density of the particles,
The pores may increase the deposition of the microparticles for example deposition of microparticles in for example the lung may be increased by about 50% or more.
The presence of the pores can result in the aerodynamic diameter of the particles being smaller than the geometric diameter, resulting in improved delivery by oral inhalation.
In the case of a powder comprising porous microparticles, the pores of the microparticles may increase the flowability of the powder
If the porous microparticles were formulated in a suspension, for example, the microparticles may remain in suspension for a longer period of time compared to non-porous microparticles.
The increased surface area of the microparticles (organic compound) may aid in improving the solubility and/or dissolution rate of the material of the microparticles.

Advantageously, composite microparticles produced in accordance with the method of the present invention may comprise one or more organic compounds. For example, each individual microparticle may comprise one or more organic compounds.

The organic compound may be one or more selected from the group comprising: Bendroflumethiazide, Betamethasone base, Betamethasone valerate, Budesonide, Formoterol fumarate, Hydrochlorothiazide, Hydroflumethiazide, Lysozyme, Para-aminosalicylic acid, Sodium cromoglycate, Sulfadiazine, Sulfadimidine, Sulfamerazine, Trypsin, Insulin, Human growth hormone, Somatotropin, Tissue plasminogen activator, Erthyropoietin, Granulocyte colony stimulating factor (G-CSF), Factor VIII, Interferon-α, Interferon-β, IL-2, Calcitonin, Monoclonal antibodies, Therapeutic proteins/peptides/polypeptides, Therapeutic proteins derived from plants, animals, or microorganisms, and recombinant versions of these products, Monoclonal antibodies, Proteins intended for therapeutic use, cytokines, interferons, enzymes, thrombolytics, and other novel proteins, Immunomodulators, Growth factors, cytokines, and monoclonal antibodies intended to mobilize, stimulate, decrease or otherwise alter the production of hematopoietic cells in vivo.

The organic compound may be a solid material.

The present invention further provides a method for preparing porous microparticles of an organic bioactive comprising the steps of:—
combining one or more bioactives with a volatile solvent system; and
drying the system thus formed to provide substantially pure porous microparticles of the bioactive or composite porous microparticles of combinations of bioactives.

Advantageously, microparticles made in accordance with the present invention may be considered substantially pure, for example the microparticles may not contain contaminants. This aspect of the invention is considered particularly advantageous for microparticles that may be used in drug delivery systems where the purity of the drug is of utmost importance.

The advantages associated with the method of producing microparticles of organic compounds discussed above may also apply to the method of making microparticles of an organic bioactive.

The bioactive may be selected from one or more of the group comprising: bendroflumethiazide, Betamethasone base, Betamethasone valerate, Budesonide, Formoterol fumarate, Hydrochlorothiazide, Hydroflumethiazide, Lysozyme, Para-aminosalicylic acid, Sodium cromoglycate, Sulfadiazine, Sulfadimidine and Sulfamerazine, alpha and beta adrenoreceptor agonists for example salbutamol, salmeterol, terbutaline, bambuterol, clenbuterol, metaproterenol, fenoterol, rimiterol, reproterol, bitolterol, tulobuterol, isoprenaline, isoproterenol and the like and their salts, anticholinergics for example ipratropium, oxitropitun and tiotropium and the like and their salts, glucocorticoids for example beclomethasone, betamethasone, budesonide, ciclesonide, formoterol, fluticasone, mometasone, triamcinolone and the like and their salts and esters, antiallergics for example nedocromil sodium and sodium cromoglycate and the like, leukotriene inhibitors and antagonists for example montelukast, pranlukast, zafirlukast and zileuton and the like, xanthines for example aminophylline, diprophylline, etofylline, proxyphylline, theobromine and theophylline and the like, anti-infectives for example tobramycin, amikacin, ciprofloxacin, gentamicin, para-aminosalicylic acid, rifampicin, isoniazid, capreomycin, acyclovir and ritonavir and the like, antihistamines for example, terfenadine, cetrizine, loratadine and the like, pain control substances for example morphine and codeine and the like and their salts, and combinations thereof.

In one embodiment the bioactive may be a protein, peptide or polypeptide, such as a protein selected from the group comprising: Lysozyme, Trypsin, Insulin, Human growth hormone, Somatotropin, Tissue plasminogen activator, Erthyropoietin, Granulocyte colony stimulating factor (G-CSF), Factor VIII, Interferon-α, Interferon-β, IL-2, Calcitonin, Monoclonal antibodies, Therapeutic proteins/peptides/polypeptides, Therapeutic proteins derived from plants, animals, or microorganisms, and recombinant versions of these products, Monoclonal antibodies, Proteins intended for therapeutic use, cytokines, interferons, enzymes, thrombolytics, and other novel proteins, Immunomodulators, Growth factors, cytokines, and monoclonal antibodies intended to mobilize, stimulate, decrease or otherwise alter the production of hematopoietic cells in vivo, and combinations thereof.

Preferably the protein may be insulin.

In some embodiments the bioactive may be a solid material.

In a further aspect the present invention also provides a method of preparing porous microparticles of a pharmaceutically acceptable excipient comprising the steps of:
combining one or more pharmaceutically acceptable excipients with a volatile solvent system; and
drying the system thus formed to provide substantially pure porous microparticles of the pharmaceutically acceptable excipient or composite porous microparticles of combinations of pharmaceutically acceptable excipients.

Microparticles of substantially pure pharmaceutically acceptable excipients may be particularly useful, for example as a carrier for active pharmaceuticals or bioagents. For example, it is envisaged that in one respect pharmaceuticals or bioagents or the like may be coated onto/loaded into microparticles of pharmaceutically acceptable excipients, such as the microparticles may act as a carrier or delivery tool for delivering a pharmaceutical or bioagent to a pre-determined target site.

The advantages associated with the method of producing microparticles of organic compounds and bioactives discussed above may also apply to the method of making microparticles of a pharmaceutically acceptable excipient.

The pharmaceutically acceptable excipient may be one or more selected from the group comprising: magnesium stearate, monosaccharides, for example glucose, galactose, fructose and the like; disaccharides, for example trehalose, maltose, lactose, sucrose and the like; trisaccharides, for example raffinose, acarbose, melezitose and the like; cyclic oligosaccharides/cyclodextrins, for example hydroxpropyl-β-cyclodextrin, hydroxyethyl-62 -cyclodextrin, α-cyclodextrin, β-cyclodextrin, γ-cyclodextin, methyl-β-cyclodextrin, dimethyl-β-cyclodextrin, sulfobutylether-β-cyclodextrin, randomly methylated-β-cyclodextrin and the like; soluble polymers, for example polyvinylpyrrolidone, for example PVP 10,000, PVP 40,000, PVP 1,300,000, polyethylene glycol and the like; sugar alcohols/polyols, for example mannitol, xylitol, sorbitol and the like; amino sugars and oligosaccharides, for example inulin and maltodextrin and the like; polysaccharides, for example starch, glycogen and the like; cellulose and cellulose derivatives, for example methylcellulose, ethylcellulose, hydroxypropylmethyl cellulose and the like; deoxy, amino and other sugar derivatives, for example deoxy-glucose, deoxy-ribose, galactosamine and the like, and combinations thereof.

The pharmaceutically acceptable excipient may be a solid material.

In one embodiment, the method of preparing porous microparticles of a pharmaceutically acceptable excipient (as described above) may further comprise the step of:
combining one or more bioactives with the pharmaceutically acceptable excipients in a volatile solvent system
   prior to the step of drying the system.

Preferably, the volatile solvent system used in accordance with the methods of the present invention may comprise a mixture of solvents.

In one embodiment one of the solvents may be water.

Preferably, the solvent system may comprise a volatile solvent such as an aliphatic hydrocarbon, an aromatic hydrocarbon, a halogenated hydrocarbon, an alcohol, an aldehyde, a ketone, an ester, an ether or mixtures thereof.

Desirably, the solvent system may comprise ethanol.

Alternatively, the solvent system may comprise methanol.

The solvent system used may depend on the properties of the organic compound and/or bioactive and/or pharmaceutically acceptable excipient used. For example, a different solvent system may be used for hydrophobic compounds/bioactives/excipients as compared to the solvent system used for hydrophilic compounds/bioactives/excipients.

The solvent system may comprise from about 5% to about 40% v/v of water, such as from about 10% to about 20% v/v of water.

In one embodiment the system may comprise a process enhancer, such as ammonium carbonate.

The process enhancer may be present in an amount of from about 5% to about 70%, such as from about 10% to about 25%.

Preferably, the system may be dried by spray drying.

In one embodiment the spray drying may be carried out in air.

In a further embodiment, the spray drying may be carried out in an inert atmosphere, such as nitrogen.

Preferably, the spray drying may be carried out at an inlet temperature of from about 30° C. to about 220° C., such as from about 70° C. to about 130° C.

Preferably, the spray drying may be carried out at an inlet temperature of from about 70 to about 110° C. for ethanol systems. Whereas the spray drying may be carried out at an inlet temperature of from about 60° C. to about 130° C. for methanol systems.

In accordance with the present invention the pores of the microparticles may range in size from about 20 to about 1000 nm, preferably the microparticles may be nanoporous.

In accordance with the present invention, the term “pore” may be understood to include gaps, voids, spaces, fissures and the like.

Desirably, the pores may be substantially spherical in shape.

The present invention may also provide for substantially pure porous microparticles of an organic compound, and/or porous microparticles comprising spherical aggregates of organic compound.

In addition, the present invention may also provide for porous microparticles comprising sponge-like particles of organic compound.

Desirably, the invention may also provide porous microparticles of organic compound comprising substantially hollow spheres with nanopores in the shell.

Advantageously, porous microparticles in accordance with the present invention may not contain a surfactant or surfactant residue.

Porous microparticles of organic compound, in accordance with the present invention, may comprise one or more selected from the group consisting: Bendroflumethiazide, Betamethasone base, Betamethasone valerate, Budesonide, Formoterol fumarate, Hydrochlorothiazide, Hydroflumethiazide Hydroxpropyl-β-cyclodextrin, Lysozyme, Para-aminosalicylic acid, PVP 10,000, PVP 40,000, PVP 1,300,000, Raffinose, Sodium cromoglycate, Sulfadiazine, Sulfadimidine, Sulfamerazine, Trehalose, Trypsin, Insulin, Human growth hormone, Somatotropin, Tissue plasminogen activator, Erthyropoietin, Granulocyte colony stimulating factor (G-CSF), Factor VIII, Interferon-α, Interferon-β, IL-2, Calcitonin, Monoclonal antibodies, Therapeutic proteins/peptides/polypeptides, Therapeutic proteins derived from plants, animals, or microorganisms, and recombinant versions of these products, Monoclonal antibodies, Proteins intended for therapeutic use, cytokines, interferons, enzymes, thrombolytics, and other novel proteins, Immunomodulators, Growth factors, cytokines, and monoclonal antibodies intended to mobilize, stimulate, decrease or otherwise alter the production of hematopoietic cells in vivo.

The present invention may also provide for substantially pure porous microparticles of organic bioactive, and/or porous microparticles comprising spherical aggregates of organic bioactive.

In addition, the present invention may provide porous microparticles comprising sponge-like particles of organic bioactive.

Desirably, multiporous micro particles of organic bioactive may comprise substantially hollow spheres with nanopores in the shell.

Advantageously, porous microparticles of organic bioactives in accordance with the present invention may not contain a surfactant or surfactant residue.

Porous micro particles of organic bioactives in accordance with the present invention may comprise one or more bioactive selected from the group comprising: Bendroflumethiazide, Betamethasone base, Betamethasone valerate, Budesonide, Formoterol fumarate, Hydrochlorothiazide, Hydroflumethiazide, Lysozyme, Para-aminosalicylic acid, Sodium cromoglycate, Sulfadiazine, Sulfadimidine and Sulfamerazine.

Desirably, the bioactive is a protein, peptide or polypeptide. For example, the protein may be one or more selected from the group comprising: Lysozyme, Trypsin, Insulin, Human growth hormone, Somatotropin, Tissue plasminogen activator, Erthyropoietin, Granulocyte colony stimulating factor (G-CSF), Factor VIII, Interferon-α, Interferon-β, IL-2, Calcitonin, Monoclonal antibodies, Therapeutic proteins/peptides/polypeptides, Therapeutic proteins derived from plants, animals, or microorganisms, and recombinant versions of these products, Monoclonal antibodies, Proteins intended for therapeutic use, cytokines, interferons, enzymes, thrombolytics, and other novel proteins, Immunomodulators, Growth factors, cytokines, and monoclonal antibodies intended to mobilize, stimulate, decrease or otherwise alter the production of hematopoietic cells in vivo.

Preferably, the protein is insulin.

The present invention may also provide porous micro particles of organic bioactive in combination with one or more excipient selected from the group comprising:

Hydroxypropyl-β-cyclodextrin, Raffinose, Trehalose, Magnesium stearate, PVP 10,000, PVP 40,000 and PVP 1,300,000.

The present invention may also provide substantially pure porous micro particles of a pharmaceutically acceptable excipient, and/or porous micro particles comprising spherical aggregates of pharmaceutically acceptable excipient.

The present invention may further provide porous microparticles comprising sponge-like particles of pharmaceutically acceptable excipient.

Desirably, multiporous microparticles of pharmaceutically acceptable excipient may comprise substantially hollow spheres with nanopores in the shell.

Preferably, porous microparticles of pharmaceutically acceptable excipient in accordance with the present invention may not contain a surfactant or surfactant residue.

Porous microparticles of pharmaceutically acceptable excipient in accordance with the present invention may comprise one or more selected from the group comprising: Hydroxypropyl-β-cyclodextrin, Raffinose, Trehalose, Magnesium stearate, PVP 10,000, PVP 40,000 and PVP 1,300,000.

The present invention may further provide for a pharmaceutical composition comprising substantially pure organic bioactive porous micro particles. Desirably the pharmaceutical composition may further comprise a pharmaceutically acceptable excipient or adjuvant.

Preferably, the pharmaceutical composition may be in the form of a powder.

In one embodiment the present invention may also provide substantially pure porous microparticles of insulin.

Furthermore the present invention may further comprise:
substantially pure porous microparticles which are as good as or better than formulations containing carrier excipient for drug delivery or dry powder by inhalation, providing a 50% (or greater) increase in fine particle fraction determined in in vitro studies;
porous microparticles which have higher solubilities than non-porous materials, providing a three-fold (or greater) increase in solubility compared to non-porous materials;
porous microparticles which have higher dissolution rates than non-porous materials, providing a three-fold (or greater) increase in dissolution rate compared to non-porous materials;
porous microparticles which have lower densities than non-porous materials, providing a three-fold (or greater) decrease in density compared to non-porous materials;
porous microparticles which have higher surface areas than non-porous materials providing a six-fold (or greater) increase in surface area compared to non-porous materials; and
porous microparticles which have lower sedimentation rates in liquid suspension than non-porous particles.

### BRIEF DESCRIPTION OF THE DRAWINGS

The invention will be more clearly understood from the following description thereof given by way of example only, in which:—

FIG. A is a schematic of a spray drying process;

FIG. 1 is an SEM of budesonide spray dried at the conditions outlined in Example 1;

FIG. 2 is an SEM of budesonide spray dried at the conditions outlined in Example 2;

FIG. 3 is a graph showing respirable fractions (i.e. deposited on stage 2 of twin impinger) of unprocessed budesonide (BRAW) and porous budesonide samples spray dried at the inlet temperatures of 78° C. and 85° C.;

FIG. 4 shows the visual suspension quality of the budesonide systems in HFA-134a after mixing (left) and after 2 minutes (right). From left: budesonide “as received” powder, spray dried budesonide, porous budesonide spray dried at the inlet temperature of 78° C. and porous budesonide spray dried at the inlet temperature of 85° C.;

FIG. 5 is an SEM of budesonide spray dried at the conditions outlined in Example 3;

FIG. 6 is a graph of respirable fractions or fine particle fractions (FPFs) for each of the aerosolised budesonide powder systems;

FIG. 7 is a graph of respirable fractions or fine particle fractions (FPFs) for each of the aerosolised budesonide and budesonide/lactose carrier blends systems;

FIG. 8 is an SEM of bendroflumethiazide (BFMT) spray dried at the conditions outlined in Example 4;

FIG. 9 is an SEM of bendroflumethiazide spray dried at the conditions outlined in Example 5;

FIG. 10 is an SEM of bendroflumethiazide spray dried at the conditions outlined in Example 6. FIG. 10a presents the system spray dried from 60% v/v ethanol and FIG. 10b presents the system spray dried from 70% v/v ethanol;

FIG. 11 is an SEM of bendroflumethiazide spray dried at the conditions outlined in Example 7;

FIG. 12 shows the comparison of the suspension stability (sedimentation rates) of (A) MDI containing micronised BFMT and (B) MDI containing NPMPs of BMFT (spray dried from 80% (v/v) ethanol). Photograph taken immediately after agitation (t=0), after four hours (t=4 h) and after 7 days (t=7 days);

FIG. 13 is an SEM of sulfadimidine spray dried at the conditions outlined in Example 11;

FIG. 14 is an SEM of sulfadimidine spray dried at the conditions outlined in Example 12;

FIG. 15 shows the surface area and bulk density of sulfadimidine systems outlined in Example 12 (FIG. 15a) and sulfamerazine systems outlined in Example 16 (FIG. 15b);

FIG. 16 is a graph showing the respirable fractions (as determined by Andersen cascade impactor) of unprocessed sulfadimidine (SRAW) and porous sulfadimidine samples spray dried at the conditions outlined in Example 11 and Example 12;

FIG. 17 is an SEM of sulfadiazine spray dried at the conditions outlined in Example 15;

FIG. 18 is an SEM of sulfamerazine spray dried at the conditions outlined in Example 16;

FIG. 19 is a graph showing the respirable fractions (as determined by Andersen cascade impactor) of unprocessed sulfamerazine and porous sulfamerazine samples spray dried at the conditions outlined in Example 16;

FIG. 20 is an SEM of sulfamerazine spray dried at the conditions outlined in Example 17;

FIG. 21 is an SEM of sodium cromoglycate spray dried at the conditions outlined in Example 19;

FIG. 22 is an SEM of sodium cromoglycate spray dried at the conditions outlined in Example 20;

FIG. 23 is a graph showing the respirable fractions (as determined by Andersen cascade impactor) of unprocessed sodium cromoglycate, non-porous spray dried system and NPMPs of sodium cromoglycate spray dried at the conditions outlined in Examples 19 and 20;

FIG. 24 is an SEM of betamethasone base spray dried at the conditions outlined in Example 21;

FIG. 25 is an SEM of betamethasone valerate spray dried at the conditions outlined in Example 22;

FIG. 26 is an SEM of para-aminosalicylic acid spray dried at the conditions outlined in Example 23;

FIG. 27 is an SEM of para-aminosalicylic acid spray dried at the conditions outlined in Example 24;

FIG. 28 is an SEM of para-aminosalicylic acid spray dried at the conditions outlined in Example 25;

FIG. 29 is an SEM of lysozyme spray dried at the conditions outlined in Example 26;

FIG. 30 is an SEM of lysozyme spray dried at the conditions outlined in Example 27;

FIG. 31 is an SEM of trypsin spray dried at the conditions outlined in Example 28;

FIG. 32 is an SEM of budesonide/formoterol fumarate spray dried at the conditions outlined in Example 29;

FIG. 33 is an SEM of bendroflumethiazide/sulfadimidine spray dried at the conditions outlined in Example 30;

FIG. 34 is an SEM of trehalose spray dried at the conditions outlined in Example 31;

FIG. 35 is an SEM of raffinose spray dried at the conditions outlined in Example 32;

FIG. 36 is an SEM of hydroxypropyl-β-cyclodextrin spray dried at the conditions outlined in Example 33;

FIG. 37 is an SEM of hydroxypropyl-β-cyclodextrin spray dried at the conditions outlined in Example 34;

FIG. 38 is an SEM of hydroxypropyl-β-cyclodextrin spray dried at the conditions outlined in Example 35;

FIG. 39 is an SEM of hydroxypropyl-β-cyclodextrin spray dried at the conditions outlined in Example 36;

FIG. 40 is an SEM of polyvinylpyrrolidone 10,000 spray dried at the conditions outlined in Example 37;

FIG. 41 is an SEM of polyvinylpyrrolidone 40,000 spray dried at the conditions outlined in Example 38;

FIG. 42 is an SEM of budesonide/hydroxypropyl-β-cyclodextrin spray dried at the conditions outlined in Example 39;

FIG. 43 are SEMs of sulfadimidine/polyvinylpyrrolidone 10,000 spray dried at the conditions outlined in Example 40. FIG. 43a presents the system containing sulfadimidine/polyvinylpyrrolidone 10,000 in the ratio 9:1 and FIG. 43b is the system containing sulfadimidine/polyvinylpyrrolidone 10,000 in the ratio 8:2;

FIG. 44 are SEMs of bendroflumethiazide/polyvinylpyrrolidone 10,000 spray dried at the conditions outlined in Example 41. FIG. 44a presents the system containing bendroflumethiazide/polyvinylpyrrolidone 10,000 in the ratio 9:1 and FIG. 44b is the system containing bendroflumethiazide/polyvinylpyrrolidone 10,000 in the ratio 1:1;

FIG. 45 is an SEM of bendroflumethiazide/magnesium stearate spray dried at the conditions outlined in Example 42;

FIG. 46 is an SEM of sulfadimidine/magnesium stearate spray dried at the conditions outlined in Example 43. FIG. 46a presents the system containing sulfadimidine/magnesium stearate in the ratio 99.5:0.5 and FIG. 46b is the system containing sulfadimidine/magnesium stearate in the ratio 99:1;

FIG. 47 is an SEM of lysozyme/hydroxypropyl-β-cyclodextrin spray dried at the conditions outlined in Example 44;

FIG. 48 is an SEM of lysozyme/trehalose spray dried at the conditions outlined in Example 45;

FIG. 49 is an SEM of lysozyme/raffinose spray dried at the conditions outlined in Example 46;

FIG. 50 is an SEM of hydrochlorothiazide/polyvinylpyrrolidone 10,000 spray dried at the conditions outlined in Example 47;

FIG. 51 is an SEM of bendroflumethiazide/hydroxypropyl-β-cyclodextrin spray dried at the conditions outlined in Example 48;

FIG. 52 is an SEM of bendroflumethiazide/polyvinylpyrrolidone 40,000 spray dried at the conditions outlined in Example 49;

FIG. 53 is an SEM of bendroflumethiazide/polyvinylpyrrolidone 1,300,000 spray dried at the conditions outlined in Example 50;

FIG. 54 is an SEM of hydroflumethiazide/polyvinylpyrrolidone 10,000 spray dried at the conditions outlined in Example 51;

FIG. 55 is an SEM hydrochlorothiazide/hydroxypropyl-β-cyclodextrin spray dried at the conditions outlined in Example 52 and

FIG. 56 is an SEM of hydroxypropyl-β-cyclodextrin/polyvinylpyrrolidone 10,000 spray dried at the conditions outlined in Example 53.

### DETAILED DESCRIPTION OF THE INVENTION

The invention provides an improved method for preparing porous microparticles. The porous microparticles may consist of an organic compound alone, such as a bioactive or pharmaceutically acceptable excipient or may comprise a combination of organic compounds for example a bioactive associated with a pharmaceutical excipient and/or adjuvant which may act to improve particle performance or as a stabiliser for the pharmaceutical. Alternatively, composite microparticles may comprise a mixture of one or more bioactive and/or one or more pharmaceutically acceptable excipient and/or one or more adjuvant or combinations thereof.

The method of the invention also provides for the preparation of porous adjuvant/excipient materials alone. These porous excipient particles may be subsequently loaded with a pharmaceutical material such as a bioactive.

In the invention a surfactant is not required and an emulsion is not formed. Typically in preparing porous microparticles a surfactant is required and may be used to stabilise the emulsion.

The invention is directed towards providing an improved process for producing porous microparticles of organic compounds and porous microparticles produced by the process.

The process of microparticle production generally involves adding an organic compound to a mixed solvent system. In most instances the mixed liquid system will consist of a solvent in which the organic compound solid is soluble and a second solvent, which is also miscible with the first solvent and in which the organic compound is less soluble. The appropriate co-solvent system containing the organic compound is atomized and dried by spray drying, and the resultant porous microparticles collected. A process enhancer, such as ammonium carbonate may be added to the mixed solvent system to promote/enhance pore formation. Any process enhancer included in the system as a solute volatilises/decomposes in the spray drying process and is thus absent from the final microparticle formed by the process.

Composite microparticles consisting of a bioactive-adjuvant and/or a bioactive-excipient combination may also be prepared. The adjuvant may be added to improve the functionality (e.g. flowability) or stability of the powder.

Porous particles of drug entities have been prepared by other methods. Pulmospheres™, for example, are porous particles produced by spray drying phospholipids-stabilised fluorocarbon-in-water emulsions (Dellamary et al., Pharm. Res. 17, 168-174 (2000). The highly volatile fluorocarbon acts as a “blowing agent” to blow holes in the solid particles.

Thou et al. (J. Materials Sci., 36, 3759-3768 (2001)) described the production of porous or honeycomb particles of the polymer, polymethyl methacrylate (PMMA), by spray drying solutions of the polymers dissolved in mixed solvent systems. The authors did not, however, apply the technique to small molecular weight organic bioactives nor did they apply it to small molecular weight organic excipient/adjuvant materials. The raw PMMA used in the study had an average molecular weight of 120,000. It is a water insoluble polymer.

Leong, similarly described the production of porous particles of inorganic materials (J. Aerosol Sci., 18, 511-524 (1987)).

Surprisingly, we have found that porous microparticles may be produced by spray drying small molecular weight organic compounds (molecular weight typically less than 1,000) and/or a combination of small molecular weight organic compounds such as bioactive and/or excipient and/or adjuvant from mixed solvent systems. Also surprisingly we have found that porous microparticles may be produced by spray drying water soluble proteins or polymers from mixed solvent systems.

Surprisingly and unexpectedly, we have found that porous microparticles may be produced by spray drying solutions (single liquid phase) rather than emulsions (two or multiphase), as previous processes for producing porous particles have employed Advantageously, with this technology pure active particles (microparticles consisting only of pure bioactive with no added excipient) or bioactive-excipient combination particles can be prepared in a one-step process.

In one embodiment of the invention, porous microparticles of bendroflumethiazide are produced by spray drying from an ethanol/water (90:10) solvent mixture.

The selection of experimental parameters such as a particular solvent mixture in a particular ratio and with appropriate spray drying conditions (temperature, feed rate, pump rate, aspirator setting), enables the production of porous microparticles of pure organic compounds. Furthermore, the process can also be employed to produce composite porous microparticles.

In the process of the invention the organic compound is dissolved in a suitable co-solvent system, i.e. a liquid consisting of a solvent in which the organic compound is soluble and a second solvent, which is also miscible with the first solvent and in which the organic compound is less soluble. Preferably the more volatile solvent should be a good solvent for the organic compound, and the less volatile solvent (i.e. that with the higher boiling point) should be a poor solvent for the organic compound (i.e. an ‘antisolvent’). The solution of the organic compound in the appropriate co-solvent system is then atomized and dried, for example by spray drying, and the resultant porous microparticles collected.

To render the two solvents miscible, a proportion of a third solvent may, in some cases be necessary. In other cases a small amount of a third solvent may be added to increase the solubility of the organic compound so as to obtain an adequate yield.

An agent (process enhancer), such as ammonium carbonate, may also be included to improve/promote pore formation or to control solvent pH.

The process enhancer, where it is employed, is removed by decomposition/volatilisation or chemical reaction in the spray drying process, thus the process results in microparticles of pure organic compound or, in the case of composite systems (e.g. bioactive and excipient), composite material consisting of only the starting solid constituents.

Nasal and pulmonary delivery offer fast rates of absorption and onset of action of drugs as well as avoiding the issue of drug degradation in the gastrointestinal tract, providing an alternative to injection. First pass metabolism is also avoided.

For oral inhalation particles must be typically <10 μm in diameter and have a narrow particle size distribution. The porous microparticles of the invention fulfil these criteria.

The microparticles of the invention are typically between about 0.5 and about 10μm in diameter, with pores/gaps/voids/spaces/fissures in the range about 5 nm to about 1000 nm, for example about 50 nm to about 1000 nm. The microparticles of the present invention can in some instances be regarded as nanoporous microparticles (NPMPs).

It is anticipated that porous microparticles in accordance with the invention have reduced interparticulate attractive forces. Porous microparticles have improved flow characteristics relative to micronised drug materials. They have low bulk densities and exhibit smaller aerodynamic diameters than represented by their geometric diameters. They have potentially improved efficiency for administration to the lungs in the dry form (dry powder inhaler formulations) and also a potential for improved suspension stability in liquid inhaler formulations (metered dose inhalers), with a reduced tendency to sediment in the liquefied propellant. The porous microparticles of the invention provide improved in vitro deposition in the Andersen Cascade impactor compared to micronised or non-porous spray dried drug.

The process is not restricted to any chemical class or pharmacological class of organic compound. The organic compound product is often amorphous on spray drying, either alone or with the aid of an ‘enhancer’ (which may have the effect of increasing the glass transition temperature (Tg), allowing formation of a stable glass at room temperature).

Processing of some materials in the manner described in the invention may result in crystalline porous microparticles.

The microparticles may have nanopores in their structure or the particles may resemble clumps or aggregates of nanosized particles, the packing of which results in nanospaces.

The morphology for the various types of porous microparticles prepared by the method of the invention is as follows. All the measurements given are based on SEM observations.

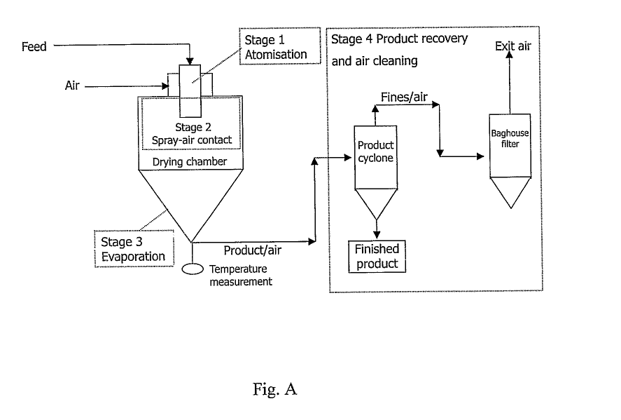

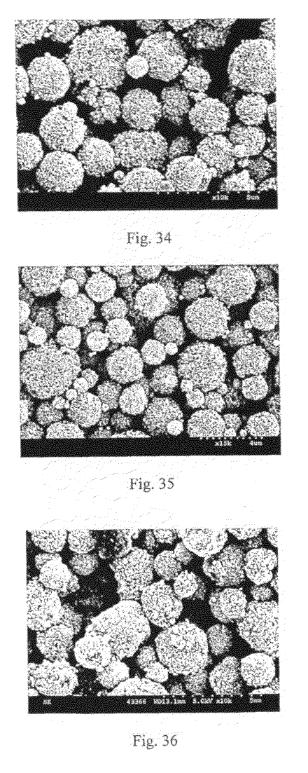

I. Particles appear as spherical formations or deformed spheres (also particles with other shapes e.g. donut-like) consisting of fused/sintered particulate structures of spherical shape. The surfaces of particles are highly irregular with visible holes ranging from 20 to 1000 nm in diameter. Examples of organic compounds presenting this type of morphology (dependant on processing conditions) are budesonide (with nanoparticulate structures ranging from 50 to 200 nm in diameter, FIGS. 1, 2), sulfadiazine (with nanoparticulate structures ranging from 50 to 200 nm in diameter), betamethasone base (FIG. 24) and betamethasone valerate (FIG. 25), budesonide/formoterol fumarate (FIG. 32) as well as trehalose (FIG. 34), raffinose (FIG. 35).

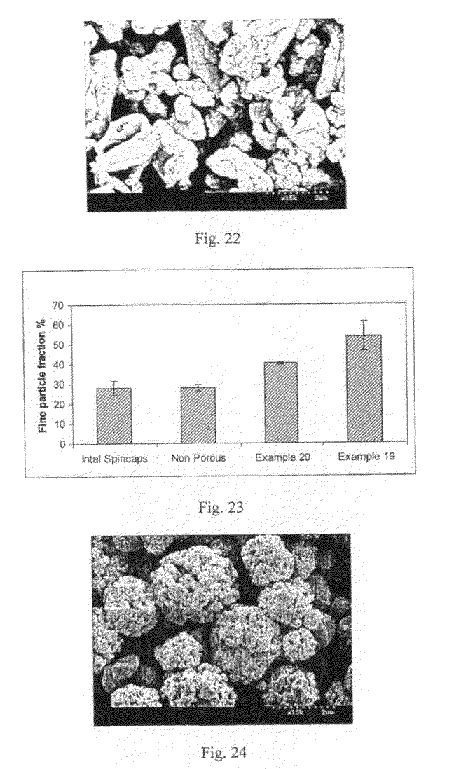

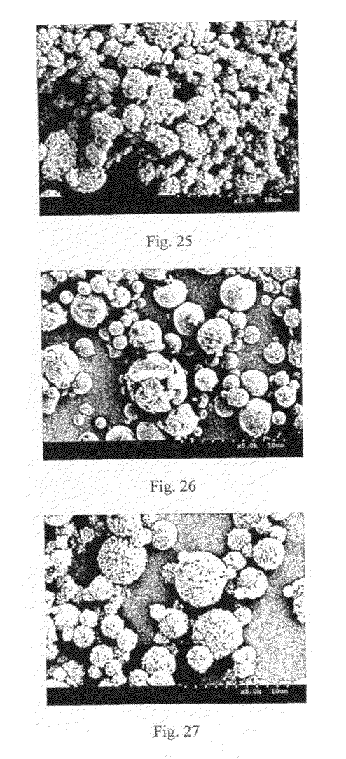

II. Particles appear as roughly spherical formations with irregular surfaces consisting of fused/sintered particulate structures. An example of an organic compound presenting this type of morphology is bendroflumethiazide (with nanoparticulate structures ranging from 50 to 300 nm in diameter, FIGS. 8, 9, 10, 11), bendroflumethiazide composite NPMPs: bendroflumethiazide/sulfadimidine (FIG. 33) bendroflumethiazide/magnesium stearate (FIG. 45) and bendroflumethiazide/PVP 1,300,000 (FIG. 53) as well as sodium cromoglycate (FIG. 22)) para-aminosalicylic acid and its complex (FIGS. 26, 27, 28) and hydroxypropyl-β-cyclodextrin (FIG. 39).

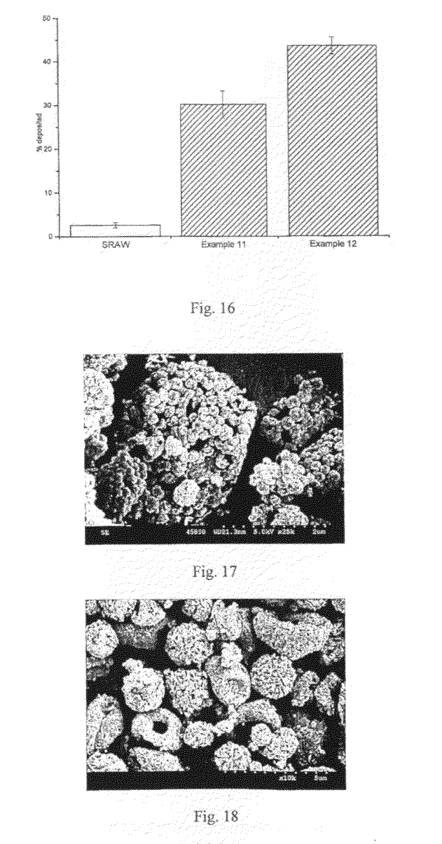

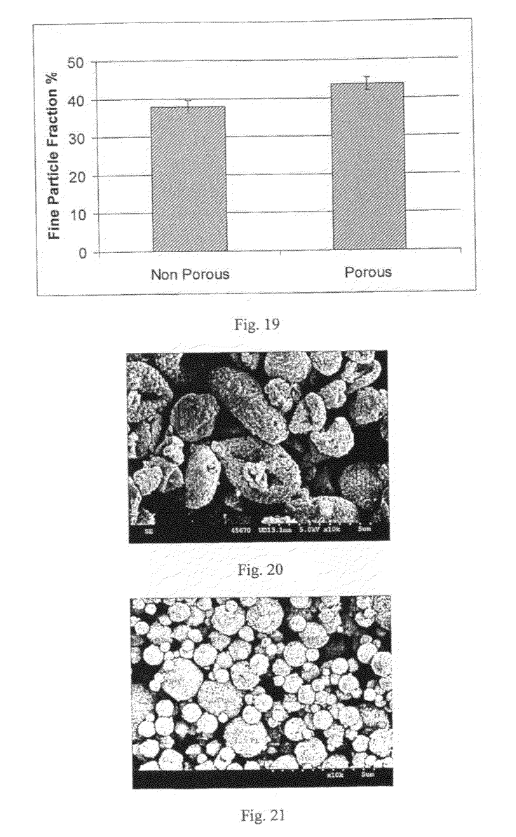

III. Particles consisting of spherical particles fused/sintered less strongly than those presented in type I or II. The spherical substructures are easily discernible and more uniform in size than those particulate substructures described as type I or II and also the connections between them are thinner than those shown in type I or II. Examples of organic compounds which have been rendered porous and present this type of morphology are sulfamerazine (with nanoparticulate structures ranging from 200 to 500 nm in diameter, FIG. 20), sulfadimidine (with nanoparticulate structures ranging from 200 to 300 nm in diameter) and sulfadiazine (with nanoparticulate structures ranging from 100 to 200 nm in diameter, FIG. 17).

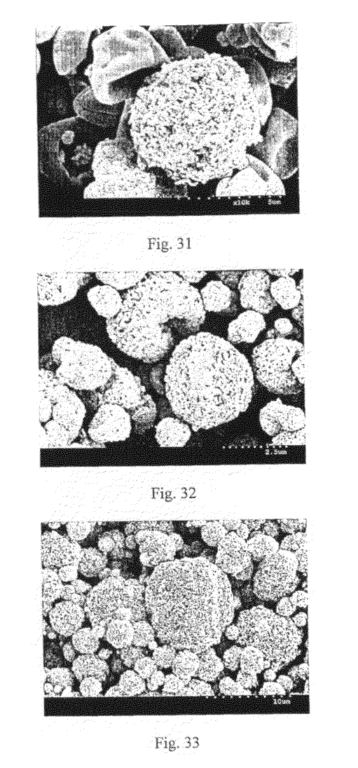

IV. Particles similar in construction to those described as type I, but consisting of particulate structures of elongated shapes. An example of a bioactive obtainable in this form is sulfamerazine (FIG. 18) and trypsin (FIG. 31).

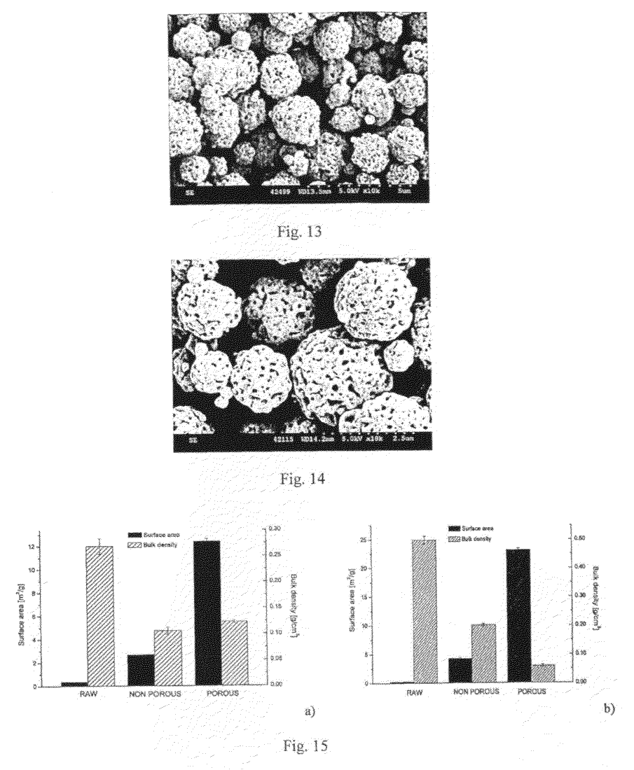

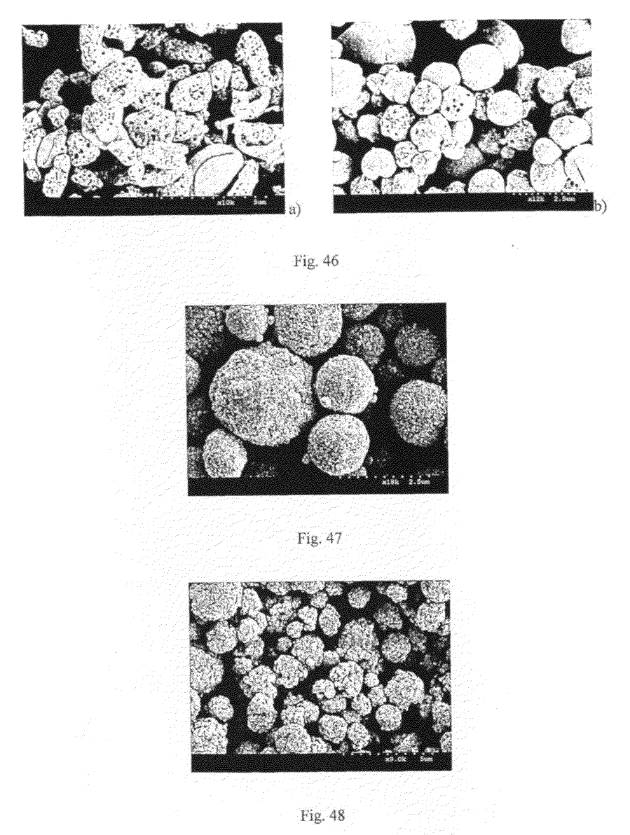

V. Spherical or deformed spheres with holes in the generally smooth surface giving the appearance of channels going through the particles. The diameter of the holes varies between 100 and 1000 nm. Examples of organic compounds obtainable in this form are budesonide (FIG. 5) and sulfadimidine (FIGS. 13, 14) and sulfadimidine/magnesium stearate porous microparticles (FIG. 46).

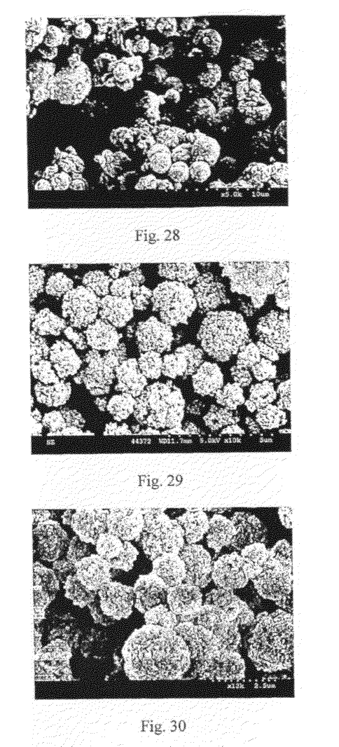

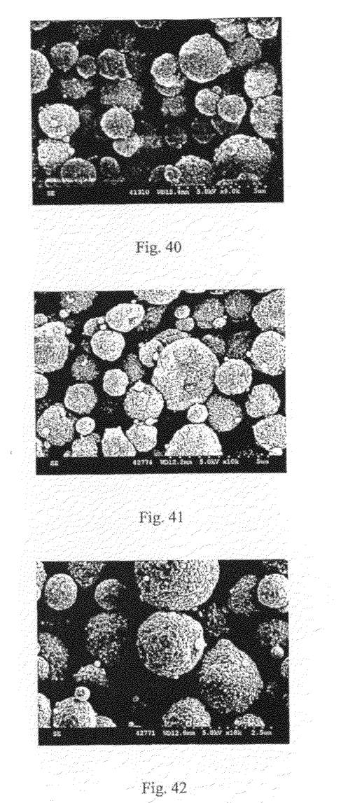

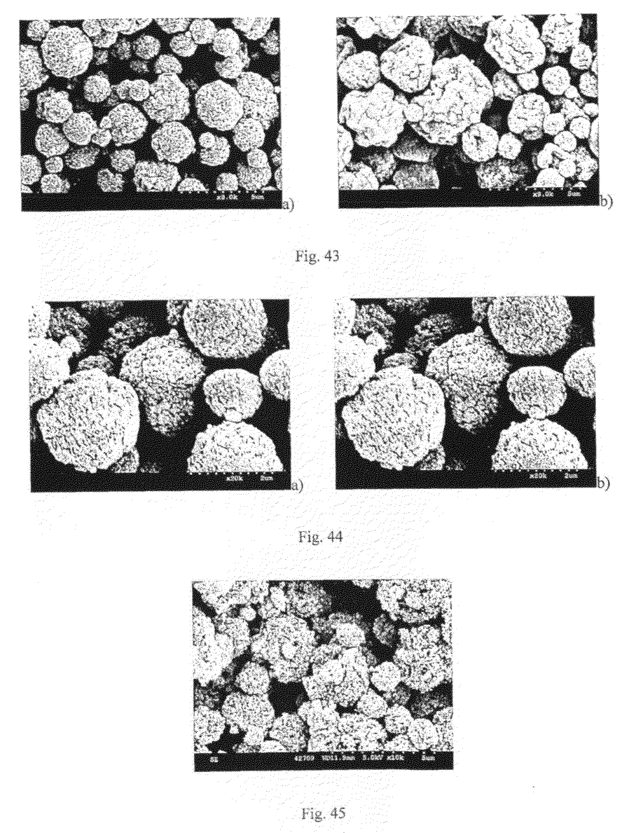

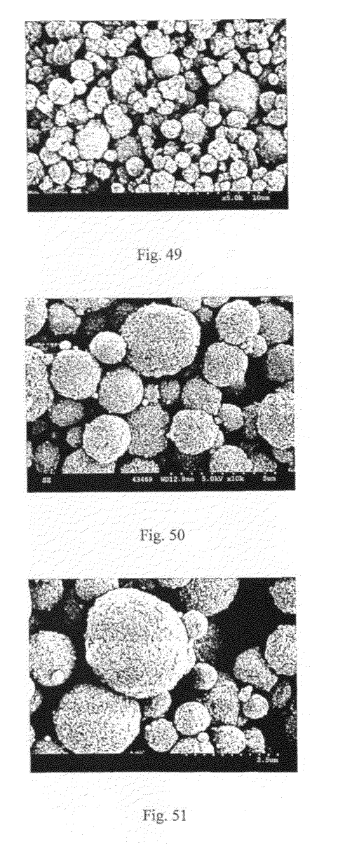

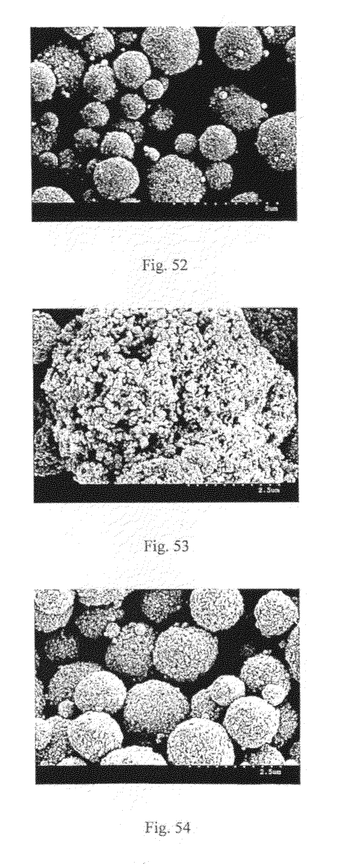

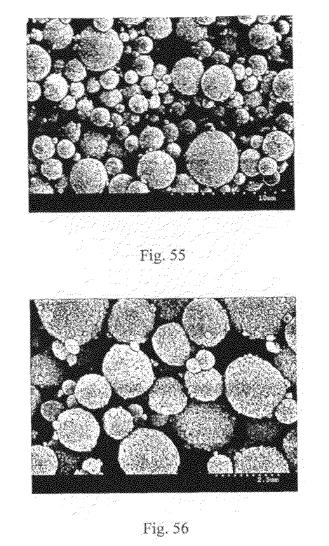

VI. Spherical or collapsed (e.g. raisin-like) particles with rough surfaces and visible holes having diameters between 10 and 50 nm. The appearance of these particles is more compact and “solid” than any of the aforementioned types of porous microparticles. Examples of organic compounds which have been rendered porous and display this type of outer morphology are sodium cromoglycate (FIG. 21), lysozyme (FIGS. 29, 30), hydroxypropyl-β-cyclodextrin (FIGS. 36, 37, 38), polyvinylpyrrolidone 10,000 (FIG. 40), polyvinylpyrrolidone 40,000 (FIG. 41), budesonide/hydroxypropyl-β-cyclodextrin (FIG. 42), sulfadimidine/polyvinylpyrrolidone 10,000 (FIG. 43), bendroflumethiazide/polyvinylpyrrolidone 10,000 (FIG. 44), lysozyme/hydroxypropyl-β-cyclodextrin (FIG. 47), lysozyme/trehalose (FIG. 48), lysozyme/raffinose (FIG. 49), hydrochlorothiazide/polyvinylpyrrolidone 10,000 (FIG. 50), benciroflumethiazide/hydroxypropyl-β-cyclodextrin (FIG. 51), bendroflumethiazide/polyvinylpyrrolidone 40,000 (FIG. 52), hydroflumethiazide/polyvinylpyrrolidone 10,000 (FIG. 54), hydrochlorothiazide/hydroxypropyl-β-cyclodextrin (FIG. 55) and polyvinylpyrrolidone 10,000/hydroxypropyl-β-cyclodextrin (FIG. 56).

The median particle size of two batches of sulfamerazine (one batch consisting mainly of particles type III and one batch a mix of particles type II and III) was 1.83 and 2.07 μm as determined by Malvern Mastersizer 2000 at the dispersant pressure 2 bar.

The median particle size of a sulfadimidine batch (made of particles type W) was 2.38 μm as determined by Malvern Mastersizer 2000 at the dispersant pressure 2 bar.

Increasingly, new drug products coming from drug discovery programmes are poorly soluble and difficult to absorb. The oral route of drug delivery is still by far the most popular and there is a need for drug delivery systems that ensure adequate dissolution and bioavailability of poorly soluble drugs. The process of the invention results in an amorphous high-energy drug form with a high porosity and therefore high surface area. These characteristics should result in improved solubility and dissolution rate and potentially improved bioavailability.

The dispersibility of a powder in liquid and the stability of suspensions for oral administration may be improved by the use of the porous microparticles of the invention, which will settle slowly in suspension due to their small particle size and low bulk density. This in turn will ensure improved and accurate dosing.

The method for preparing porous microparticles of the invention preferably utilises a spray drying technique. Any similar process involving atomisation followed by solvent removal could be used. Spray drying involves the conversion of a liquid solution or suspension to a solid powder in a one-step process. Referring to FIG. A, a spray dryer consists of a feed delivery system, an atomizer, heated air supply, drying chamber, solid-gas separators e.g. cyclone separator (primary collection) and product collection systems: cyclone separator, drying chamber & filter bag collectors (secondary collection). The spray drying process consists of four steps: (1) atomisation of the liquid feed, (2) droplet-gas mixing, (3) removal of solvent vapour and (4) collection of dry product.

While spray drying is typically used to produce porous microparticles, it is anticipated that they may also be produced by similar technologies involving atomisation of the liquid system followed by solvent removal.

The invention employs a novel spray drying process to produce porous microparticles of organic compounds. The organic compounds may be organic bioactives alone, organic adjuvants/excipients alone, organic bioactives in combination with adjuvants and/or excipients or combinations of organic adjuvants/excipients

The adjuvants or excipients may include sugars and non-polymeric excipients. The porous excipient microparticles may be first formed and then combined with a pharmaceutical or bioactive.

The porous microparticles of the invention may be prepared by dissolving the organic compound in a solution of a suitable solvent mixture such as:
2) water/ethanol
3) water/ethanol/ammonium carbonate
4) water/methanol/ammonium carbonate
5) water/methanol/n-butyl acetate
6) methanol/n-butyl acetate
   and subsequently spray drying the solution thus formed.

Other solvent combinations that may be used in the process of obtaining porous microparticles:
1) water/methanol
2) water/ethanol/ammonium hydrogen carbonate
3) water/ethanol/ammonium acetate
4) water/ethanol/ammonium formate
5) water/ethanol/chloral hydrate
6) water/ethanol/menthol
7) methanol/n-propyl acetate
8) methanol/isopropyl acetate

In general, for hydrophobic organic compounds the following solvent mixtures appear to be more suitable:
1) water/ethanol
2) water/methanol
3) water/ethanol/ammonium carbonate
4) water/ethanol/ammonium hydrogen carbonate
5) water/ethanol/ammonium acetate
6) water/ethanol/ammonium formate
7) water/methanol/ammonium carbonate
8) water/ethanol/chloral hydrate
9) water/ethanol/menthol

In general, for hydrophilic organic compounds the following solvent mixtures appear to be more suitable:
1) water/methanol/n-butyl acetate
2) methanol/n-butyl acetate
3) methanol/n-propyl acetate
4) methanol/isopropylacetate

The actual solvent combination used depends on the physicochemical properties of the organic compound. One of the solvents should preferably be a volatile solvent for the organic compound while another should be a less volatile antisolvent.

While most porous microparticles are prepared from mixed solvent systems it may also be possible to obtain porous microparticles from single solvent systems. The porous microparticles thus prepared may be crystalline in nature.

Other volatile solvents (apart from ethanol and methanol) that may be used in the process of the invention for spray drying to produce porous microparticles are:
Hydrocarbons e.g hexane. heptane, octane, nonane, decane, 2-pentene, 1-hexene, 2-hexene and their isomers.
Halogenated hydrocarbons e.g. dichloromethane, chloroform, ethyl chloride, trichloroethylene
Aromatic hydrocarbons and their derivatives e.g. benzene, toluene, xylene, cresol, ethylbenzene, chlorobenzene, aniline
Cyclic hydrocarbons and heterocyclic solvents e.g. cyclopentane, cyclohexane, tetrahydrofuran, pyrrolidine, 1,4-dioxan
Alcohols e.g. 1-propanol, 2-propanol, 1-butanol, 2-butanol, tert-butanol, pentyl alcohols, 2-chloroethanol, ethyl glycol
Aldehydes e.g. ethanal, propionaldehyde, butanal, 2-methylbutanal, benzaldehyde
Ketones e.g. acetone, methylethyl ketone, 2-pentanone, 2-hexanone
Esters e.g. ethyl acetate, propyl acetate, isopropyl acetate, butyl acetate
Ethers e.g. dipropyl ether, tert-amyl ethyl ether, butyl ethyl ether, tert-butyl methyl ether, butyl ether, pentyl ether

The porous microparticles of the invention have potential application in preparations for oral and nasal inhalation and for oral drug delivery.

In the examples below, we describe a process which renders the following bioactives porous:
Bendroflumethiazide
Betamethasone base
Betamethasone valerate
Budesonide
Lysozyme
Para-aminosalicylic acid
Sodium cromoglycate
Sulfadiazine
Sulfadimidine
Sulfamerazine
Trypsin
Hydroflumethiazide
Formoterol fumarate
Hydrochlorothiazide

In the examples below, we describe a process which renders the following bioactive combinations porous:
Budesonide/formoterol fumarate
Bendroflumethiazide/sulfadimidine

The process described also renders the following adjuvants/excipients porous
Hydroxypropyl-β-cyclodextrin
Trehalose
PVP 10,000
PVP 40,000
Raffinose
Magnesium stearate
PVP 1,300,000

In the examples below, we describe a process which renders the following bioactives and adjuvants/excipient combinations porous:
Hydroxypropyl-β-cyclodextrin/budesonide mixed system
Hydroxypropyl-β-cyclodextrin/bendroflumethiazide mixed system
Hydroxypropyl-β-cyclodextrin/hydrochlorothiazide mixed system
Hydroxypropyl-β-cyclodextrin/PVP 10,000 mixed system
PVP 10,000/bendroflumethiazide mixed system
PVP 10,000/sulfadimidine mixed system
PVP 10,000/hydroflumethiazide mixed system
PVP 10,000/hydrochlorothiazide mixed system
PVP 40,000/bendroflumethiazide mixed system
PVP 1,300,000/bendroflumethiazide mixed system
Bendroflumethiazide/magnesium stearate mixed system
Sulfadimidine/magnesium stearate mixed system
Lysozyme/hydroxypropyl-β-cyclodextrin mixed system
Lysozyme/trehalose mixed system
Lysozyme/raffinose mixed system
Budenoside/formoterol fumarate dihydrate mixed system
Bendroflumethiazide/sulfadimidine mixed system

The following is a list of substances that may potentially act as process enhancers:
Ammonium carbonate
Ammonium acetate
Ammonium benzoate
Ammonium formate
Ammonium hydrogen carbonate
Ammonium chloride
Ammonium bromide
Ammonium perchlorate
Ammonium dithiocarbamate
Ammonium thiosulphate and other ammonium salts
Camphor
Chloral hydrate
Menthol

Porous microparticle technology provides significant advantages over other porous particle technologies, some of the advantages are summarised below:
Porous microparticles can be produced from solutions rather than from two phase emulsion systems. The emulsion systems must contain a surfactant or emulsion stabiliser. Such stabilisers will remain as a residual/contaminant in the porous particles prepared, with potential for toxicity. For instance, lung lesions were observed in the bronchi to alveoli after a single intratracheal instillation of polyoxyethylene 9 lauryl ether (Laureth-9) and sodium glycocholate in rats (Suzuki, et al., J. Toxic. Sci. 25, 49-55 (2000)). Toxicological studies carried out by Li et al. indicated that charge-inducing agents e.g. stearylamine and diacetylphosphate may cause an apparent disruption of pulmonary epithelial cells (Pharm. Res., 13, 76-79 (1996)). Wollmer et al. suggest that repeated administration of surface active agents may include lung water accumulation and development of atelectasis (Pharm. Res., 17, 38-41 (2000)).
   In their assessment of Èxubera™, a dry powder inhalable form of insulin, a FDA advisory committee expressed concern about excipients in Exubera's formulation, which members feared could irritate the lungs (AAPS Newsmagazine, 9(1), 13 (2006)).
With our technology there is no requirement to include a surfactant for the purpose of producing porous particles. Particles can therefore be produced that consist only of pure organic compound.
The process itself of preparing typically a solution of organic compound in the mixed solvent system is much simpler and potentially less time-consuming and therefore less expensive than the emulsion approach. The issue of physical instability of emulsions (phase separation and sedimentation) is also avoided.
The simpler process involves fewer operations/manipulations than other production processes for porous particles and there are therefore fewer sources of variability and potentially improved reproducibility associated with the novel process.
The technology may be used to prepare composite porous microparticles also. Porous microparticles have been prepared which consist of a bioactive entity along with a stabilising agent, bioactive (drug) penetration enhancer (to improve absorption) or lubricant (to facilitate removal from the inhaler device). Thus an excipient (additive) can be included in the formulation without an additional processing step.

Potential Applications of Porous Microparticles

Pulmonary Drug Delivery

Porous particles are known to be beneficial for drug delivery to the respiratory tract by oral inhalation. Porous microparticles have reduced interparticulate attractive forces and improved flow characteristics relative to micronised drug materials. They have low bulk densities and exhibit smaller aerodynamic diameters than represented by their geometric diameters, facilitating greater deposition in the lower pulmonary region, as is required for systemic drug delivery—of particular importance for the delivery of proteins, such as insulin. They have potential for improved efficiency of administration to the lungs in the dry form (dry powder inhaler formulations) and also a potential for improved suspension stability in liquid inhaler formulations (metered dose inhalers), with a reduced tendency to sediment in the liquefied propellant.

There is an increasing interest in recent years in the pulmonary route as an alternative to the parenteral route for the delivery of protein-based biopharmaceuticals. Recently, a spray dried form of insulin (with excipients in a buffered sugar-based matrix) has been marketed for delivery of the bioactive by the pulmonary route (White et al., Exubera®: Pharmaceutical Development of a Novel Product for Pulmonary Delivery, Diabetes Technology and Therapeutics, 7(6) 896-906 (2005)). In their assessment of Èxubera™, an FDA advisory committee expressed concern about excipients in Exubera's formulation, which members feared could irritate the lungs (AAPS Newsmagazine, 9(1), 13 (2006)). NPMPs in accordance with the present invention offer the potential for porous protein, peptide or polypeptide particles suitable for inhalation which contain no excipient materials.

Porous microparticles technology may be applied to such protein, peptide or polypeptide actives to increase the efficacy of the formulation.

In the present invention, trypsin and lysozyme have been employed to illustrate that pure nanoporous microparticles can be produced from a protein/polypeptide/peptide material.

Oral Drug Delivery

The increased porosity associated with porous microparticles will be reflected in an increased powder surface area. Increasingly new drug products coming from drug discovery programmes are poorly soluble and difficult to absorb. There is a high attrition rate of new chemical entities in the early stages of drug design and drug development projects because of problems with poor solubility. The oral route of drug delivery is still by far the most popular and there is a need for drug delivery systems that ensure adequate dissolution and bioavailability of poorly soluble drugs. An increased porosity and powder surface area are likely to result in an increased dissolution rate. If the drug is also present in a high energy amorphous form, this may result in an improved solubility, dissolution rate and potentially improved bioavailability.

The novel spray drying process we propose typically results in an amorphous high-energy drug form with a high porosity and therefore high surface area. These characteristics are likely to result in improved solubility and dissolution rate and potentially improved bioavailability.

The stability of suspensions for oral administration may be improved by the use of porous microparticles, which will settle slowly in suspension due to their small particle size and low bulk density. This in turn will ensure improved and accurate dosing.

The invention will be more clearly understood from the following examples thereof.

Experimental

Spray Drying

All systems were spray dried using a Büchi B-191 or Büchi B-290 Mini Spray Dryer (Büchi Laboratoriums-Technik AG, Switzerland).

The B-191 operates only in the suction mode (or open mode) i.e. a negative pressure is formed in the apparatus and the drying medium employed was compressed air.

The B-290 spray dryer can be used either in the suction (open) mode (with compressed air or nitrogen) or in the closed (blowing) mode. The closed mode was used when the Büchi Inert Loop B-295 was attached. This accessory enables the safe use of organic solvents in a closed loop and nitrogen was used as the drying gas.

When an ethanol/water or methanol/water mixture was used as the solvent for the process, only the concentration of the organic solvent is given e.g. 95% v/v ethanol indicates that the solvent was made of 95% v/v ethanol and 5% v/v deionised water.

Differential Scanning Calorimetry (DSC)

DSC experiments were conducted using a Mettler Toledo DSC 821^{e} with a refrigerated cooling system (LabPlant RP-100). Nitrogen was used as the purge gas. Hermetically sealed aluminium pans with three vent holes were used throughout the study and sample weights varied between 4 and 10 mg. DSC measurements were carried out at a heating/cooling rate of 10° C./min. The DSC system was controlled by Mettler Toledo STAR^{e} software (version 6.10) working on a Windows NT operating system.

Thermogravimetric Analysis (TGA)

TGA was performed using a Mettler TG 50 module linked to a Mettler MT5 balance. Sample weights between 5 and 12 mg were used and placed into open aluminium pans. A heating rate of 10° C./min was implemented in all measurements. Analysis was carried out in the furnace under nitrogen purge and monitored by Mettler Toledo STAR^{e} software (version 6.10) with a Windows NT operating system.

Scanning Electron Microscopy (SEM)

Visualisation of particle size and morphology was achieved by scanning electron microscopy (SEM). Scanning electron micrographs of powder samples were taken using a Hitachi S-4300N (Hitachi Scientific Instruments Ltd., Japan) variable pressure scanning electron microscope. The dry powder samples were fixed on an aluminium stub with double-sided adhesive tabs and a 10 nm thick gold film was sputter coated on the samples before visualisation. The images were formed from the collection of secondary electrons.

Fourier Transform Infrared Spectroscopy (FTIR)

Fourier Transform infrared Spectroscopy (FTIR) was carried out using a Nicolet Magna IR 560 E.S.P. spectrophotometer equipped with MCT/A detector, working under Omnic software version 4.1. Potassium bromide (KBr) discs were prepared based on 1% w/w sample loading. Discs were prepared by grinding the sample with KBr in an agate mortar and pestle, placing the sample in an evacuable KBr die and applying 8 tons of pressure, in an IR press. A spectral range of 650-4000 cm⁻¹, resolution 2 cm⁻¹ and accumulation of 64 scans were used in order to obtain good quality spectra.

Powder X-Ray Diffraction (XRD)

Powder X-ray diffraction measurements (XRD) were made on samples in low background silicon mounts, which consisted of cavities 0.5 mm deep and 9 mm in diameter (Bruker AXS, UK). A Siemens D500 Diffractometer was used. This consists of a DACO MP wide-range goniometer with a 1.0° dispersion slit, a 1.0° anti-scatter slit and a 0.15° receiving slit. The Cu anode X-ray tube was operated at 40 kV and 30 mA in combination with a Ni filter to give monochromatic CuKα X-rays (λ=1.54056). Measurements were taken from 5° to 40° C. on the theta 2 scale at a step size of 0.05° per second for qualitative analysis.

Particle Size Measurement

The particle size distribution of the powder samples was determined by laser diffraction using the Malvern Mastersizer 2000 (Malvern Instruments Ltd., Worcs., U.K.) with the Scirocco 2000 accessory. The dispersive air pressure range employed was from 1.0-3.5 bar. Samples were generally run at a vibration feed rate of 50%. The particle size was given as d(0.5), which is the median particle size of volume distribution. This value states the particle size corresponding to the 50% point on the cumulative percent undersize curve and will be referred to here as the, median diameter (MD), in μm. Mastersizer 2000 software (Version 5.22) was used for analysis of the particle size.

Density Measurements

Bulk density (bρ) was measured by filling the dry powder in a 1 ml graduated syringe (Lennox Laboratory supplies, Naas Rd. Dublin 12) with a funnel. The weight of the powder required to fill the 1 ml graduated syringe was recorded to calculate bρ. The tap density (tρ) of the powder was then evaluated by tapping the syringe onto a level surface at a height of one inch, 100 times. The resultant volume was recorded to calculate tρ. Each measurement was performed in triplicate.

The Carr's compressibility index of some of the systems was calculated from the following equation:

compressibility index (%)=[(tap density−bulk density)/tap density]×100

Lower values of the index are desirable as they indicate better flow.

Surface Area Analysis

Surface area analysis was performed using a Micromeritics Gemini 2370 Surface Area Analyser with nitrogen as the adsorptive gas. Samples were degassed using a Micromeritics FlowPrep 060 Degasser. The Flowprep uses a flowing gas (nitrogen) which is passed over a heated sample to remove moisture and other contaminants. All raw materials were degassed for 24 hrs at 40° C. Processed samples following spray drying were degassed at 25° C. for 24 hrs. BET multipoint surface areas were determined. The volume of nitrogen adsorbed at six relative pressure points between 0.05 and 0.3 was measured. The BET multipoint area was calculated using either five or six of the measured points (whichever results gave the highest correlation coefficient). Analyses were performed at least in duplicate.

Solubility Studies

A. Sealed Ampoule Method

Saturated solubility studies were determined in water and 1% w/v PVP at 37° C., by the sealed ampoule method (Mooney et al., J. Pharm. Sci., 70 (1981) 13-22). Excess solid (approximately 2-3 times the estimated solubility of raw, spray dried non-porous and spray dried porous material) was place in 10 ml solvent in a glass ampoule and the ampoule was heat sealed. Ampoules were placed in a shaker water bath, at 37° C. for 24 or 48 hours. After 24 hours the ampoule was opened and a 5 ml sample withdrawn and filtered through a 0.45 μm membrane filter. After 48 hours a sample was taken from a second ampoule and treated similarly. The concentration of the material was determined by UV spectroscopy of a suitable dilution of the filtered sample. Solubility determinations were done in triplicate, the quoted solubilities being the average of the three results.

B. Overhead Stirrer Method

Dynamic solubility studies were determined by the overhead stirrer method. This apparatus was used to determine the saturated solubility profile of the material over time. The solubility vessel consisted of a water-jacketed flat-bottomed 50 ml cylindrical glass vessel. The system was maintained at 37° C. by means of a Heto thermostat pumping motor and water bath. The medium (water or 1% w/v PVP) was introduced into the vessel at the start of the run. Excess solid (approximately 2-3 times the estimated solubility of raw, spray dried non-porous and spray dried porous material) was placed in the medium in the vessel. The medium was stirred using an overhead stirrer. 2 ml samples were removed at appropriate intervals up to 24 hours from a zone midway between the base of the vessel and the surface of the medium. Samples were filtered through a 0.45 μm membrane filter. All runs were performed in triplicate, the quoted values being the average of the three results. Samples were analyzed by UV spectroscopy of a suitable dilution of the filtered sample.

Suspension Sedimentation Analysis

Sedimentation analysis was carried out on suspensions of bendroflumethiazide (BFMT) and sulfadimidine. 25 ml suspensions were prepared by mixing water and Tween 80 (96:4 v/v) with 150 mg of the drug powder. The suspensions were transferred to 25 ml graduated cylinders, mixed thoroughly and their sedimentation observed over time.

Preparation of MDI Systems

In order to prepare metered dose inhalers, 20 mg of powder was weighed into glass vials. Afterwards a 25 μl metering valve (Bespak, UK) was crimped onto the glass vial and the liquid propellant HFA-134a was added through the nozzle. The final weight of each MDI (without the container and metering valve) was 10 g. The last two steps were performed using a Pamasol P 2016 aerosol filling station (Pamasol Willi Mader AG, Pfäffikon, Switzerland). Prepared MDIs were homogenised in a Bransonic 220 ultrasonic bath (UK) for 1 min.

Solid State Stability Study

Solid state stability studies were conducted at two different conditions of temperature and humidity according to ICH protocol (ICH, 2003). The systems were placed in weighing boats in glass chambers containing saturated solutions of
NaBr to maintain a constant relative humidity of 60% for long-term testing
NaCl to maintain a constant relative humidity of 75% for accelerated testing

The glass chamber containing the NaBr solution was stored at 25° C. and the glass chamber containing NaCl solution was stored at 40° C. in incubators (Gallenkamp, UK). At appropriate time intervals samples of each solid material was removed from the ovens and analysed where appropriate.

In Vitro Dry Powder Inhaler Deposition Measurements and Aerodynamic Particle-Size Analysis Using a Cascade Impactor

The pulmonary deposition of the dry powders was investigated using an Andersen Cascade Impactor (ACI) (1 ACFM Eight Stage Non-Viable Cascade Impactor, Graseby Andersen, Atlanta, Ga.). The ACI was assembled as outlined in the United States Pharmacopoeia (U.S.P.), apparatus 3 for DPIs. Size 3 hard gelatin capsules (Farillon Ltd., U.K.) were filled to approximately 50% with the dry powder (approximately 25 mg of powder). Capsules were placed in a Handihaler™ (GlaxoSmithKline) or Spinhaler™ (Rhone Poulenc Rorer) dry powder inhaler and the liberated powder was drawn through the ACI operated at a flow rate of 28.3 l/min for 10 seconds, 48 l/min for 5 seconds or 60 l/min for 4 seconds. The amount of powder deposited on each stage of the impactor was determined by weight, UV analysis or HPLC analysis. The “emitted dose” was determined as the percent of total particle mass exiting the capsule and the “respirable fraction” or “fine particle fraction” (FPF) of the aerosolised powder calculated by dividing the powder mass recovered from the terminal stages (≦cut-off aerodynamic diameter of ˜5 μm) of the impactor by the total particle mass recovered in the impactor. A plot of the amount of powder deposited on each stage of the impactor against the effective cut-off diameter for that stage allowed calculation of the (experimental) mass median aerodynamic diameter (MMAD) of the particles and also the calculation of the geometric standard deviation (GSD). Results reported are the average of at least three determinations.

In Vitro Aerosol Characterisation Using a Twin Stage Impinger

The apparatus used was a twin stage impinger conforming to the specification in the British Pharmacopoeia (2004) and European Pharmacopoeia (2004).

The powders were aerosolized using a dry powder inhalation device (Rotahaler®, Allen & Hanburys, U.K.). The aerodynamic particle deposition was investigated using the twin impinger (Model TI-2, Copley) containing 7 and 30 ml of 80% v/v ethanol for stage 1 and 2, respectively. A total of 50±1 mg of powder (35±2 mg for the porous budesonide systems) was loaded into a No. 3 hard gelatin capsule. After the Rotahaler® was connected to the mouthpiece of the twin impinger, a capsule was placed in the holder of the device. An air stream of 60 l/min was produced throughout the system by attaching the outlet of the twin impinger to a vacuum pump for 3 s. The drug in stages 1 and 2, mouthpiece and device was collected by rinsing with fresh solvent. The rinsed solutions were diluted to appropriate volumes, filtered through 0.45 μm PVDF filters (Millipore) and the drug contents were determined by an appropriate HPLC method. Results reported are the average of at least three determinations.

Ammonia Assay

A commercial enzymatic ammonia assay kit from Sigma (product code AA0100) was used. It is based on the reaction of ammonia with α-ketoglutaric acid (KGA) and reduced nicotinamide adenine dinucleotide phosphate (NADPH) in the presence of L-glutamate dehydrogenase (GDH). Due to oxidation of NADPH, a decrease in absorbance at 340 nm is observed and it is proportional to the ammonia concentration. The calibration curve was prepared with ammonium carbonate solution.

Budesonide (A Steroid)

### Example 1

2.5 g budesonide was dissolved in 250 ml of 80% v/v ethanol. The concentration of this mixture was equal to 1% w/v. The solution was spray dried using a Büchi B-290 Mini Spray Dryer working in the suction mode with compressed air. The process parameters are outlined below:
Inlet temperature: 78° C.
Outlet temperature: 49-50° C.
Aspirator setting: 100%
Airflow rate: 4 cm (670 Nl/h)
Pump setting: 30% (480 ml/h)

The SEM micrograph for budesonide spray dried from 80% v/v ethanol is shown in FIG. 1. From the SEM analysis it was estimated that the NPMPs of this system had a size distribution ranging from 1 to 6 μm. The absence of crystallinity in the system was evident from the lack of peaks on the X-ray diffractogram. A relaxation endotherm indicative of glass transition with an onset temperature at approximately 90° C. was visible followed by an exotherm (recrystallisation of the amorphous phase) with an onset temperature at approximately 120° C. and then the melting endotherm, which had an onset temperature at approximately 263° C. Particle size analysis was performed at 2 bar air pressure. The MD was determined to be 3.41 μm. Particle size analysis of the system was also carried out at different air pressures (1, 2 and 3.5 bar). A shift of particle size distribution to the low size range was observed, with a dramatic change in the percentage volume of particles in the nanoparticle (<1 μm) size range observed as a result of increasing pressure. The percentage particles in the nanoparticle size range was determined to be 6.67% at 1 bar pressure whereas at 3.5 bar there was 11.33% of the particles <1 μm. A corresponding decrease in the MD was also observed, with a MD of 4.59 μm at 1 bar and a MD of 2.87 μm at 3.5 bar. The bulk (bρ) and tap (tp) densities of this system were calculated to be 0.08 g/cm³ and 0.14 g/cm³, respectively.

Also, another batch of budesonide NPMPs was produced from 80% v/v at the above conditions, but containing 15% ammonium carbonate (by total weight of dissolved solids). The bulk and tap densities of these NPMPs were calculated to be 0.09 g/cm³ and 0.17 g/cm³, respectively. These densities were lower than that determined for raw crystalline budesonide (bp and tp of 0.18 g/cm³ and 0.30 g/cm³, respectively) and also were much lower than that measured for the smooth non-porous amorphous spheres of budesonide spray dried from 95% v/v ethanol (bp and tp of 0.13 g/cm³ and 0.26 g/cm³, respectively).

Overall, nanoporous microparticles of budesonide were obtained with a Büchi B-290 Mini Spray Dryer working in the suction mode with compressed air when the following conditions were utilised:
80% v/v ethanol
0% and 15% ammonium carbonate (by total weight of dissolved solids)
1% w/v concentration of the feed solution
78° C. inlet temperature
100% aspirator setting
670 Nl/h drying medium throughput
30% pump setting

### Example 2

1.08 g budesonide was dissolved in 145 ml of 80% v/v ethanol using an ultrasonic bath, and then 0.12 g ammonium carbonate (which constituted 10% by weight of solids) was added to the clear solution of budesonide and mixed using a magnetic stirrer until the salt crystals had completely dissolved. The total weight of solids dissolved in the ethanol was 1.2 g, which gave a solution concentration equal to 0.83% w/v. The solution was spray dried using a Büchi B-191 Mini Spray Dryer with a compressed air supply.

The process parameters are outlined below:
Inlet temperature: 78° C.
Outlet temperature: 57-58° C.
Aspirator setting: 85% (−27 mbar)
Airflow rate: 600 Nl/h
Pump setting: 15% (218 ml/h)

A small endotherm assigned to the T_{g} of budesonide was observed in the DSC trace and the midpoint determined was ˜91° C. This temperature corresponds well to that of the T_{g} of amorphous budesonide, estimated to be ˜89.5° C. The budesonide main recrystallisation exotherm occurred at ˜116° C. and just prior to this a second, low in magnitude exotherm peaked at ˜102° C. The melting endotherm was sharp with a peak at ˜262° C. Infrared analysis was carried out on the co-spray dried sample to confirm if all ammonium carbonate was removed during drying. The spectrum perfectly matched the absorption spectrum of spray dried budesonide alone and even minor changes in either peak positions or shapes were absent.

No thermal events of ammonium carbonate were seen for both co-spray dried systems, indicating, as supported by the FTIR analysis, that the powder was composed solely of amorphous budesonide.

The amorphous nature of the powder was confirmed by a diffused “halo” appearing on the X-ray diffractogram. A sample SEM micrograph of the budesonide co-spray dried with ammonium carbonate system is shown in FIG. 2. The sample consisted exclusively of spheroidal porous particles. The non-solid structure of the particle was confirmed when the powder was viewed at a higher magnification.

A second batch of budesonide was spray dried at similar conditions as outlined in Example 1 but the inlet temperature used was 85° C. The powder obtained consisted of a mixture of porous and “wrinkled”, corrugated particles having rough surfaces.

The particle size distribution profiles (measured at 3 bar air pressure) of the above systems were different and the sample spray dried at 85° C. showed a narrower particle size distribution. The system processed at the inlet temperature of 78° C. exhibited a fraction of submicron particles. Similar values of the median particle size (measured at 3 bar air pressure) were obtained and were 2.9 and 2.6 μm for the system spray dried at 78° C. and 85° C., respectively.

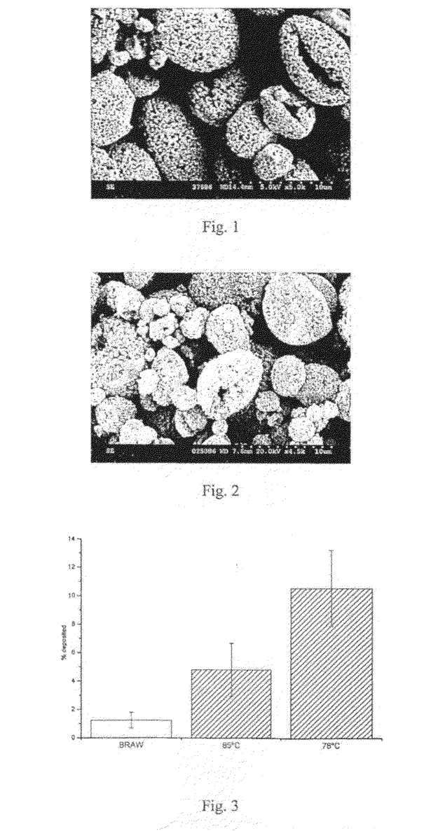

The respirable fractions, measured with the use of a twin impinger apparatus, achieved from the two powders consisting of nanoporous budesonide particles were significantly different with better performance of the sample processed at 78° C. All fine particle fractions attained with the porous particles of budesonide were significantly greater (10.5% and 4.8% for 78° C. and 85° C., respectively) than the fine particle fraction determined for micronised, crystalline budesonide (1.6%) (FIG. 3).

The two batches of the nanoporous microparticle budesonide powders were also prepared as suspension MDIs. Compared with the crystalline drug, less floc formation was observed and more even suspensions were produced (see FIG. 4). Budesonide smooth spheres (non-porous) spray dried from 95% v/v ethanol formed larger particle agglomerates consisting of caked powder and visible flocs which sedimented the fastest. The sedimentation rate of unprocessed budesonide and porous budesonide spray dried at the inlet temperature of 78° C. were comparable with the latter sedimenting slightly slower. This effect can be attributed to the lower bulk density of the porous sample because this material had a significantly greater median particle size (3.4 μm) than budesonide “as received” (1.4 μm) and yet sedimented more slowly.

Overall, nanoporous microparticles of budesonide were obtained with a Büchi B-191 Mini Spray Dryer when the following conditions were utilised:
80% v/v ethanol
10% and 15% ammonium carbonate (by total weight of dissolved solids)
0.77% and 1% w/v concentration of the feed solution
78° C. and 85° C. inlet temperature
85% aspirator setting
600 Nl/h drying medium throughput
15% pump setting

### Example 3

2.125 g budesonide was dissolved in 250 ml of 80% v/v methanol and then 0.375 g of ammonium carbonate (which constituted 15% by weight of solids) was added to the clear solution of budesonide and mixed using a magnetic stirrer until the powder had completely dissolved. The total weight of solids dissolved was 2.5 g which gave a solution concentration equal to 1% w/v. The solution was spray dried using a Büchi B-290 Mini Spray Dryer working in the closed mode. The drying gas utilised was nitrogen.

The process parameters are outlined below:
Inlet temperature: 70° C.
Outlet temperature: 45-48° C.
Aspirator setting: 100%
Airflow rate: 4 cm (670 Nl/h)
Pump setting: 30% (480 ml/h)

When the powder collected from the spray dryer was viewed using SEM, it was observed that all of the particles produced were porous (FIG. 5). The SEM micrograph shows that the NPMPs spray dried from 80% v/v methanol were visually much more compact than the NPMPs produced from ethanolic mixtures (Examples 1 and 2).

The spray drying of this system resulted in an amorphous product as evidenced by the absence of peaks and presence of a diffuse halo in the XRD scan. The amorphous material recrystallised on heating as evidenced by the exotherm in the DSC scan, which had an onset temperature at approximately 124° C. Prior to this exotherm a small endotherm was visible at approximately 90° C. (at higher magnification), which may be attributed to the glass transition. The recrystallisation exotherm was then followed by the melting endotherm, which had an onset temperature at approximately 260° C. FTIR indicated that the ammonium carbonate was removed during the spray drying process. The MD was determined to be 1.9 μm. The particle size analysis confirmed that the particle size distribution was much narrower for this system compared to the previous NPMPs (described in Examples 1-2). When particle size analysis of the system was carried out at different air pressures (1, 2 and 3.5 bar) the system showed no significant increase in the percentage volume of particles in the submicron size range with the increasing pressure. The bulk and tap densities of the powder were calculated to be 0.16 g/cm³ and 0.30 g/cm³ respectively. These densities are higher than that previously measured for NPMPs of budesonide and slightly lower than those measured for the raw micronised budesonide (bp and tp of 0.18 g/cm³ and 0.30 g/cm³ respectively).

Overall, nanoporous microparticles of budesonide were obtained with a Büchi B-290 Mini Spray Dryer working in the closed mode with compressed nitrogen when the following conditions were utilised:
80% and 90% v/v methanol
15% ammonium carbonate (by total weight of dissolved solids)
1% w/v concentration of the feed solution
70° C. inlet temperature
100% aspirator setting
670 Nl/h drying medium throughput
30% pump setting

The aerosolisation properties of porous budesonide particles were evaluated and compared to the drug in its micronised form and also the spray dried non-porous form. These aerosolisation properties were investigated using an Andersen Cascade Impactor. The pulmonary deposition of the following systems was determined:
Micronised budesonide
Budesonide spray dried from 95% v/v ethanol (powder consisted of smooth, spherical, non-porous particles)
Budesonide/ammonium carbonate 85:15 system spray dried from 80% v/v ethanol (spray dried at the same conditions as listed in Example 1)
Budesonide spray dried from 80% v/v ethanol (Example 1)
Budesonide/ammonium carbonate 85:15 system spray dried from 80% v/v methanol (Example 3)

The respirable fraction or fine particle fraction (FPF) for each of these aerosolised powder systems was calculated by dividing the powder mass recovered from the terminal stages (≦cut-off aerodynamic diameter 4.7 μm) of the impactor by the total particle mass recovered in the impactor. Also the values of mass median aerodynamic diameter (MMAD) and geometric standard deviation (GSD) were calculated for the above budesonide systems and are presented in Table 1.

TABLE 1

| | MMAD | GSD |
|---|---|---|
| System | (μm) | (μm) |
| Micronised budesonide | 3.67 | 3.28 |
| Budesonide spray dried from 95% v/v ethanol | 3.01 | 2.75 |
| Budesonide/ammonium carbonate 85:15 system | 3.17 | 2.27 |
| spray dried from 80% v/v ethanol | | |
| Budesonide spray dried from 80% v/v ethanol | 2.52 | 2.25 |
| Budesonide/ammonium carbonate 85:15 system | 2.47 | 2.42 |
| spray dried from 80% v/v methanol | | |

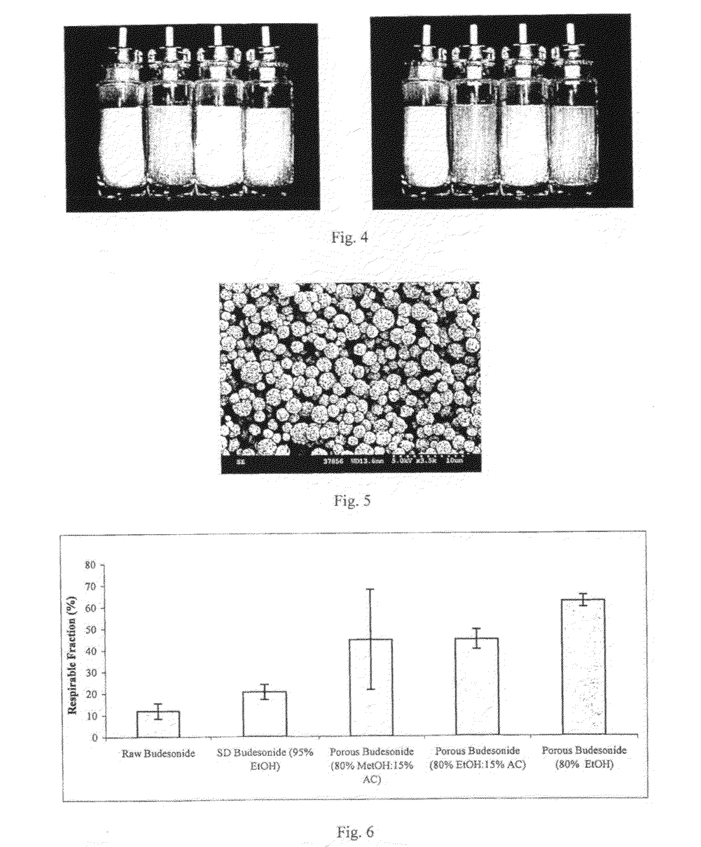

A respirable fraction or fine particle fraction (FPF) of 11.96% was determined for the raw micronised budesonide. For the budesonide system spray dried from 95% v/v ethanol, the FPF was determined to be 20.58%. For the budesonide/ammonium carbonate 85:15 system spray dried from 80% v/v ethanol, an average respirable fraction of 44.69% was achieved, demonstrating an almost four fold increase in deep lung deposition (characterised by in vitro deposition using ACI) in comparison to the micronised form of the drug. ACI experiments resulted in an average respirable fraction of 62.32% being determined for the porous powder particles of the budesonide system spray dried from 80% v/v ethanol (without process enhancer). For the four systems mentioned above, the results reported are the average of five determinations. For each system, the results obtained were consistent as can be seen from the error bars in the plot of the average respirable fractions shown in FIG. 6. In the case of the budesonide/ammonium carbonate 85:15 system spray dried from 80% v/v methanol the results were more variable and the overall respirable fraction was determined to be 44.61%.

Aerosolisation properties of various budesonide/lactose carrier blends were also investigated using an Andersen Cascade Impactor. The following systems were investigated:
Micronised budesonide
Budesonide spray dried from 95% v/v ethanol in the closed mode (powder consisted of smooth; spherical, non-porous particles)
Non-porous budesonide/lactose carrier blend mixed in the ratio 1:33.5 w/w
Budesonide NPMPs (spray dried using conditions as outlined in Example 1)
Budesonide NPMPs (spray dried using conditions as outlined in Example 1)/lactose carrier blend mixed in the ratio 1:33.5 w/w
Budesonide NPMPs (spray dried using conditions as outlined in Example 1)/lactose carrier blend mixed in the ratio 1:67.5 w/w

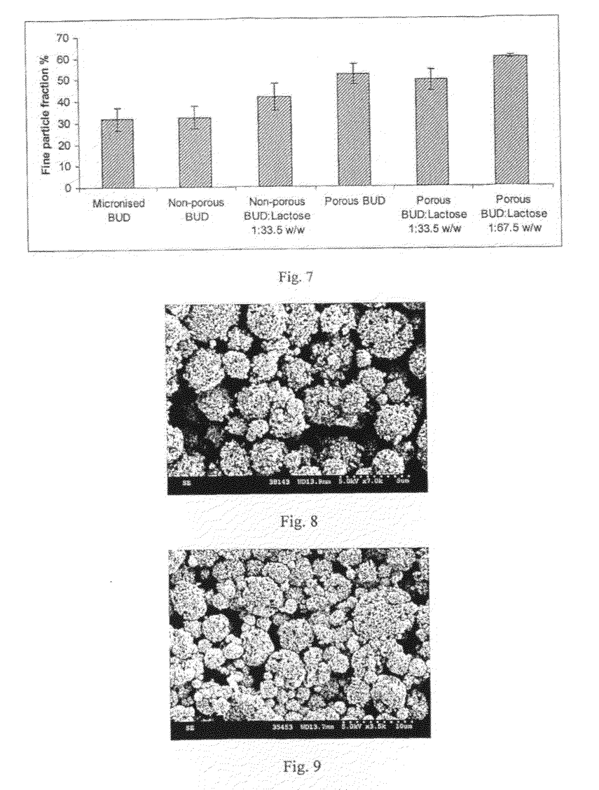

The fine particle fractions obtained from each of the powders listed above were determined to be following: 31.8±5.1 μm, 32.4±5.3 μm, 41.7±6.2 μm, 52.0±4.7 μm, 49.3±4.9 μm and 57.3±4.1 μm for micronised budesonide, non-porous spray dried drug, the blend of non-porous budesonide and lactose carrier 1:33.5 w/w, budesonide NPMPs, the blend of budesonide NPMPs and lactose carrier 1:33.5 w/w and the blend of budesonide NPMPs and lactose carrier 1:67.5 w/w, respectively. FIG. 7 presents the results graphically.

Bendroflumethiazide (BFMT) (A Bioactive)

### Example 4

2.5 g bendroflumethiazide was dissolved in 100 ml of 80% v/v ethanol. The concentration of this mixture was equal to 2.5% w/v. The solution was spray dried using a Büchi B-290 Mini Spray Dryer working in the suction mode with compressed air.

The process parameters are outlined below:
Inlet temperature: 78° C.
Outlet temperature: 51-53° C.
Aspirator setting: 100%
Airflow rate: 4 cm (670 Nl/h)
Pump setting: 30% (480 ml/h)

The collected powder consisted of nanoporous microparticles as viewed by SEM (FIG. 8). The XRD scan showed the absence of crystallinity of this system. The amorphous structure of the NPMPs was supported by the DSC data. A relaxation endotherm indicative of glass transition (T_{g}) with an onset temperature at approximately 120° C. was visible followed by an exothenn (recrystallisation of the amorphous phase) and then the melting endotherm, which had an onset temperature at approximately 219° C. Particle size analysis (at 2 bar air pressure) of the system was performed and the MD was determined to be 2.15 μm. Particle size analysis was also carried out at different air pressures (1, 2 and 3.5 bar). The percentage volume of particles in the nanoparticle size range (<1 μm) was seen to increase with the increasing pressure. The percentage volume of particles <1 μm at 3.5 bar pressure was determined to be 16.10%, in contrast to 13.06% at 2 bar and 10.45% at 1 bar pressure. A corresponding decrease in the MD was also observed at increasing pressures. The MD of the powder particles was determined to be 3.56 μm at 1 bar, 2.84 μm at 2 bar and 2.12 μm at 3.5 bar. The bulk and tap densities of this batch of NPMPs were calculated to be 0.12 g/cm³ and 0.23 g/cm³, respectively. The by and tp of the raw BFMT was calculated to be 0.29 g/cm³ and 0.58 g/cm³ respectively. For BFMT spray dried from 95% v/v ethanol and consisting of smooth spheres, the by was 0.21 g/cm³ and the tp was 0.41 g/cm³.

Overall, nanoporous microparticles of bendroflumethiazide were obtained with a Büchi B-290 Mini Spray Dryer working in the suction mode with compressed air when the following conditions were utilised:
80% v/v ethanol
0%, 10% and 15% ammonium carbonate (by total weight of dissolved solids)
0.5%, 1%, 2%, 2.5%, 2.8% and 4% w/v concentration of the feed solution
78° C. and 85° C. inlet temperature
100% aspirator setting
670 Nl/h drying medium throughput
30% pump setting
   and
90% v/v ethanol
0% and 15% ammonium carbonate (by total weight of dissolved solids)
2.5% w/v concentration of the feed solution
78° C. inlet temperature
100% aspirator setting
670 Nl/h drying medium throughput
30% pump setting

Also, nanoporous microparticles of bendroflumethiazide were obtained with a Büchi B-290 Mini Spray Dryer working in the suction mode with compressed nitrogen when the following conditions were utilised:
80% v/v ethanol
0% and 15% ammonium carbonate (by total weight of dissolved solids)
2% and 2.5% w/v concentration of the feed solution
78° C., 80° C. and 85° C. inlet temperature
100% aspirator setting
670 Nl/h drying medium throughput
30% pump setting

### Example 5

1.125 g bendroflumethiazide was dissolved in 50 ml of 80% v/v ethanol and then 0.125 g ammonium carbonate (which constituted 10% by weight of solids) was added to the clear solution of bendroflumethiazide and mixed using a magnetic stirrer until the powder had completely dissolved. The total weight of solids dissolved was 1.25 g, which gave a solution concentration equal to 2.5% w/v. The solution was spray dried using a Büchi B-191 Mini Spray Dryer using compressed air as the drying medium. The process parameters are outlined below:
Inlet temperature: 85° C.
Outlet temperature: 61° C.
Aspirator setting: 85% (−27 mbar)
Airflow rate: 600 Nl/h
Pump setting: 15% (218 ml/h)

The SEM micrograph of the NPMPs is shown in FIG. 9. The absence of crystallinity in the spray-dried system was evident from the lack of peaks. DSC supported the amorphous structure of the NPMPs. Although there was no obvious relaxation endotherm indicative of the glass transition temperature (T_{g}), a change in the baseline of the DSC trace with an onset temperature at approximately 120° C. was visible followed by an exotherm (recrystallisation of the amorphous phase) within an onset temperature at approximately 155° C., which suggests glass transition. This was then followed by the melting endotherm, which had an onset temperature at approximately 224° C. FTIR analysis of the system indicated that the ammonium carbonate was removed during the spray drying process. Particle size analysis was performed at 2 bar air pressure and the median particle size of the system was 2.6 μm. The particle size distribution was unimodal in contrast with that of the raw micronised drug, which was bimodal. Particle size analysis of the porous system was also carried out at different air pressures (1, 2 and 3.5 bar). The percentage volume of particles in the nanoparticle size range (<1 μm) was seen to increase dramatically with the increasing pressure. The percentage volume of particles <1 μm at 3.5 bar pressure was determined to be 13.59%, in contrast to 11.89% at 1 bar pressure. A corresponding decrease in the MD was also observed at increasing pressures. The MD of the powder particles was determined to be 2.64 μm at 1 bar and 1.96 μm at 3.5 bar. This contrasts with the particle size distribution of BFMT spray dried from 95% v/v ethanol (consisting of smooth spherical particles), which remained constant when subjected to increasing pressures, with no increase in the percentage volume of particles in the submicron size range evident. The bulk (bρ) and tap (tp) densities of the various BFMT systems were also different. The bp and tp of the raw BFMT was calculated to be 0.29 g/cm³ and 0.58 g/cm³ respectively. For BFMT spray dried from 95% v/v ethanol, the bp was 0.21 g/cm³ and the tp was 0.41 g/cm³. The porous particles had however a much lower bp and tp of 0.13 g/cm³ and 0.24 g/cm³ respectively.

Overall, nanoporous microparticles of bendroflumethiazide were obtained with a Büchi B-191 Mini Spray Dryer when the following conditions were utilised:
80% v/v ethanol
0%, 5%, 10%, 15% and 20% ammonium carbonate (by total weight of dissolved solids)
1.28% and 2.5% w/v concentration of the feed solution
78° C. and 85° C. inlet temperature
85% and 100% aspirator setting
600 Nl/h drying medium throughput
15% and 20% pump setting

### Example 6

1.875 g bendroflumethiazide was dissolved in 100 ml of 60% v/v ethanol and then 0.625 g ammonium carbonate (which constituted 25% by weight of solids) was added to the clear solution of bendroflumethiazide and mixed using a magnetic stirrer until the powder had completely dissolved. The total weight of solids dissolved was 2.5 g, which gave a solution concentration equal to 2.5% w/v. The solution was spray dried using a B-290 Mini Spray Dryer working in the closed mode with compressed nitrogen as the drying medium.

The process parameters are outlined below:
Inlet temperature: 110° C.
Outlet temperature: 61° C.
Aspirator setting: 100%
Airflow rate: 4 cm (670 Nl/h)
Pump setting: 30% (480 ml/h)

Also, nanoporous microparticles of bendroflumethiazide were obtained with a Büchi B-290 Mini Spray Dryer working in the closed mode with compressed nitrogen when the following conditions were utilised:
70% v/v ethanol
25% ammonium carbonate (by total weight of dissolved solids)
2.5% w/v concentration of the feed solution
110° C. inlet temperature
100% aspirator setting
670 Nl/h drying medium throughput
30% pump setting

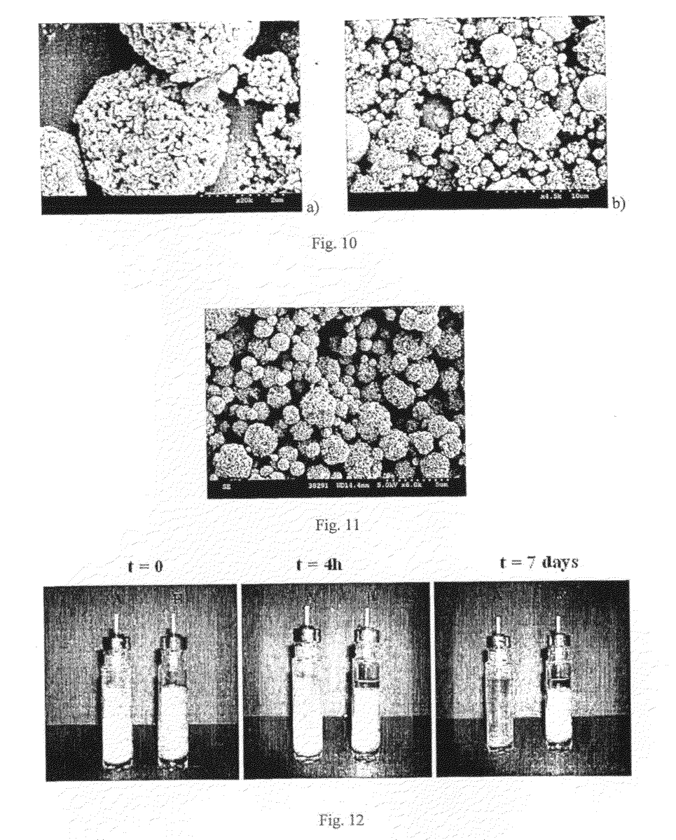

Examples of the SEM micrographs are presented in FIG. 10a for the system manufactured from 60% v/v ethanol and FIG. 10b for the system manufactured from 70% ethanol. For the latter batch of porous particles, a median particle size (by volume) was determined to be 2.5 μm. The particle size was predominantly monomodal. The percentage volume of particles in the submicron size range (less than 1 μm) was above 15%.

In order to quantify the amount of residual ammonium carbonate, the ammonia assay was carried out as described in the Experimental section on the BFMT batch spray dried from 70% v/v ethanol. The ammonia content in the sample was established to be less than 0.1% w/w.

Additionally, it has been noticed that a mixture of NPMPs and non-porous bendroflumethiazide were obtained when the following conditions were used:
80% v/v ethanol
2.5% w/v concentration of the feed solution
110° C. inlet temperature
100% aspirator setting
670 Nl/h drying medium throughput
30% pump setting

The type of spray dryer used in this experiment was a Büchi B-290 Mini Spray Dryer working in the closed mode with compressed nitrogen.

### Example 7

1.5938 g bendroflumethiazide was dissolved in 75 ml of 80% v/v methanol and then 0.2813 g ammonium carbonate (which constituted 15% by weight of solids) was added to the solution of bendroflumethiazide and mixed using a magnetic stirrer until a clear solution was obtained. The total weight of solids dissolved was 1.875 g, which gave a solution concentration equal to 2.5% w/v. The solution was spray dried using a Büchi B-290 Mini Spray Dryer working in the closed mode. The drying gas utilised was nitrogen.

The process parameters are outlined below:
Inlet temperature: 110° C.
Outlet temperature: 74° C.
Aspirator setting: 100%
Airflow rate: 4 cm (670 Nl/h)
Pump setting: 30% (480 ml/h)

When the collected powder was viewed using SEM (FIG. 11), it was observed that the particles were nanoporous in structure.

The system was amorphous, as evidenced by a broad “halo” in the XRD diffractogram. There was no obvious relaxation endotherm indicative of the glass transition temperature, however, a change in the baseline of the DSC trace with an onset temperature at approximately 120° C. was observed. This change in the baseline was followed by a recrystallisation exotherm with an onset at approximately 151° C. This was then followed by the melting endotherm, which had an onset temperature at approximately 209° C. FTIR analysis of the system indicated that the ammonium carbonate was removed during the spray drying process. The median particle size was 2.2 μm. When particle size analysis of the system was carried out at different air pressures (1, 2 and 3.5 bar), the percentage volume of particles in the nanoparticle size range (less than 1 μm) was not seen to increase significantly with the increasing pressure. The bulk and tap densities of were calculated to be 0.16 g/cm³ and 0.32 g/cm³, respectively.

Overall, nanoporous microparticles of bendroflumethiazide were obtained with a Büchi B-290 Mini Spray Dryer working in the closed mode with compressed nitrogen when the following conditions were utilised:
60% and 75% v/v methanol
15% ammonium carbonate (by total weight of dissolved solids)
1% and 2.5% w/v concentration of the feed solution
70° C. inlet temperature
100% aspirator setting
670 Nl/h drying medium throughput
30% pump setting
   and
80% v/v methanol
15% and 30% ammonium carbonate (by total weight of dissolved solids)
0.5%, 1% and 2.5% w/v concentration of the feed solution
70° C., 90° C. and 110° C. inlet temperature
100% aspirator setting
670 Nl/h drying medium throughput
30% pump setting

### Example 8

The effect of changing the process enhancer employed in the spray dried systems was also investigated. The alternative process enhancers employed were chloral hydrate and menthol.

A solution of BFMT/chloral hydrate 85:15 were spray dried from 80% v/v ethanol. The process resulted in irregular collapsed/doughnut shaped porous particles.

A 2.5% w/v solution of BFMT/menthol 85:15 was spray dried from 80% v/v ethanol. The powder produced consisted of predominantly non-porous spherical shaped particles with some irregular shaped, collapsed porous particles also present. These porous particles were morphologically different (smaller pores, collapsed particles) to those produced from the systems where ammonium carbonate was employed as the process enhancer.

### Example 9

The porous particles of the BFMT system spray dried from 80% v/v ethanol (as detailed in Example 4) were selected for formulation as a suspension for oral administration. The MD of this powder was determined to be 2.2 μm and the powder had a bulk density of 0.12 g/cm³. The stability of the BFMT NPMPs in suspension was compared to the stability of both crystalline micronised drug BFMT and also amorphous smooth spheres of BFMT spray dried from 95% v/v ethanol. 25 ml suspensions of the 3 systems were prepared as described in the Experimental Section. To assess the physical stability of the different suspensions, the sedimentation of the powder particles in the water/Tween 80 solutions were observed and compared. The powder particles of the raw BFMT and BFMT spray dried from 95% v/v ethanol were seen to completely settle in a matter of seconds. In the suspension of porous BFMT particles, it was observed after a period of four hours that while some of the particles had settled at the bottom of the graduated cylinder and some were floating at the top of the suspension that a large proportion of the porous particles remained in suspension.

### Example 10

Although BFMT is not used in inhalation therapy, its suspension stability in MDI formulations was investigated. The MDI formulations based on the same porous batch of BFMT particles as used in Example 8 and on the raw material BFMT were prepared as stated in Experimental Section. Whereas noticeable sedimentation was observed for the raw micronised drug after 4 hours, little sedimentation was observed for the NPMPs suspension over the same period of time. Indeed after a period of seven days, while the powder in the MDI containing the micronised BFMT had completely settled in the propellant, the NPMPs of BFMT in the second MDI still showed only minimal sedimentation (FIG. 12).

Sulfadimidine (A Bioactive)

### Example 11

1.5 g sulfadimidine was dissolved in 250 ml of 80% v/v ethanol using an ultrasonic bath. The drug concentration in the solution was equal to 0.6% w/v. The solution was spray dried using a Büchi B-290 Mini Spray Dryer working in the suction mode. The drying gas utilised was nitrogen.

The process parameters were employed as outlined below:
Inlet temperature: 78° C.
Outlet temperature: 47° C.
Aspirator setting: 100%
Airflow rate: 4 cm (670 Nl/h)
Pump setting: 30% (480 ml/h)

The collected powder consisted of slightly deformed spherical particles. All of them had porous structures. The powder was viewed under SEM and the micrograph is shown in FIG. 13.

Additionally, nanoporous microparticles of sulfadimidine were obtained with a Büchi B-191 Mini Spray Dryer when the following conditions were utilised:
80% v/v ethanol
10% ammonium carbonate (by total weight of dissolved solids)
0.36% w/v concentration of the feed solution
78° C. inlet temperature
85% aspirator setting
600 Nl/h drying medium throughput
15% pump setting

### Example 12

0.27 g sulfadimidine was dissolved in 100 ml of 90% v/v ethanol using an ultrasonic bath, and then 0.03 g ammonium carbonate (which constituted 10% by weight of solids) was added to the clear solution of sulfadimidine and mixed using a magnetic stirrer until the salt crystals had completely dissolved. The total weight of solids dissolved was 0.3 g, which gave a solution concentration equal to 0.3% w/v. The solution was spray dried using a Büchi B-290 Mini Spray Dryer working in the suction mode. The drying gas utilised was nitrogen.

The process parameters were employed as outlined below:
Inlet temperature: 78° C.
Outlet temperature: 49° C.
Aspirator setting: 100%
Airflow rate: 4 cm (670 Nl/h)
Pump setting: 30% (480 ml/h)

The collected powder consisted of spherical particles having evidently porous exteriors. The powder was viewed under SEM and the micrograph is shown in FIG. 14.

Overall, nanoporous microparticles of sulfadimidine were obtained with a Büchi B-290 Mini Spray Dryer working in the suction mode with compressed air when the following conditions were utilised:
80% v/v ethanol
0% and 10% ammonium carbonate (by total weight of dissolved solids)
0.3% w/v concentration of the feed solution
78° C. and 85° C. inlet temperature
100% aspirator setting
670 Nl/h drying medium throughput
30% pump setting
   and
90% v/v ethanol
0% ammonium carbonate (by total weight of dissolved solids)
0.3% w/v concentration of the feed solution
78° C. inlet temperature
100% aspirator setting
670 Nl/h drying medium throughput
30% pump setting

Also, nanoporous microparticles of sulfadimidine were obtained with a Büchi B-290 Mini Spray Dryer working in the suction mode with compressed nitrogen when the following conditions were utilised:
70% and 75% v/v ethanol
10% ammonium carbonate (by total weight of dissolved solids)
0.3% w/v concentration of the feed solution
78° C. and 85° C. inlet temperature
100% aspirator setting
670 Nl/h drying medium throughput
30% pump setting
   and
80% v/v ethanol
0%, 10% and 30% ammonium carbonate (by total weight of dissolved solids)
0.3%, 0.6%, 0.66% and 1% w/v concentration of the feed solution
78° C., 85° C., 90° C. and 95° C. inlet temperature
100% aspirator setting
670 Nl/h drying medium throughput
30% pump setting
   and
85% v/v ethanol
10% ammonium carbonate (by total weight of dissolved solids)
0.3% and 0.6% w/v concentration of the feed solution
78° C., 85° C., 90° C. and 95° C. inlet temperature
100% aspirator setting
670 Nl/h drying medium throughput
30% pump setting
   and
90% v/v ethanol
0% and 10% ammonium carbonate (by total weight of dissolved solids)
0.3% w/v concentration of the feed solution
78° C., 85° C., 90° C. and 95° C. inlet temperature
100% aspirator setting
670 Nl/h drying medium throughput
30% pump setting
   and
95% v/v ethanol
0% and 10% ammonium carbonate (by total weight of dissolved solids)
0.3% w/v concentration of the feed solution
78° C. and 90° C. inlet temperature
100% aspirator setting
670 Nl/h drying medium throughput
30% pump setting

Additionally, nanoporous microparticles of sulfadimidine were obtained with a Büchi B-290 Mini Spray Dryer working in the closed mode with compressed nitrogen when the following conditions were utilised:
70% v/v methanol
15% ammonium carbonate (by total weight of dissolved solids)
0.6% w/v concentration of the feed solution
90° C. inlet temperature
100% aspirator setting
670 Nl/h drying medium throughput
30% pump setting
   and
80% v/v methanol
15% ammonium carbonate (by total weight of dissolved solids)
0.6% w/v concentration of the feed solution
90° C. and 110° C. inlet temperature
100% aspirator setting
670 Nl/h drying medium throughput
30% pump setting

FIG. 15a displays the surface area and bulk density results measured for the raw sulfadimidine powder, non-porous drug (prepared as outlined in Example 12 but with the spray dryer set to the closed mode) and NPMPs produced as per Example 12. Generally, the lower the bulk density, the higher is the specific surface area. For the NPMPs the surface area measured 12.37±0.26 m²/g and bulk density was 0.123±0.002 g/cm³.

For the NPMPs from Example 11 a surface area of 9.41±0.06 m²/g was measured. A bulk density of 0.086±0.004 g/cm³ was determined, which is smaller than that determined for the NPMPs produced from 90% v/v ethanol.

The compressibility index for sulfadimidine was determined to be 50.3%, 51.7% and 38.4% for the raw drug powder, non-porous drug (prepared as outlined in Example 12 but with the spray dryer set to the closed mode) and NPMPs produced as per Example 12, respectively. The compressibility index for the NPMPs is significantly smaller than that measured for both the raw and non-porous material, indicating its improved flowability. NPMPs produced as per Example 11 measured a compressibility index of 43.4%, similarly lower than that measured for both raw and non-porous sulfadimidine. The respirable fractions, measured with the use of an Andersen cascade impactor, achieved from the two powders spray dried in Example 11 and Example 12 were not significantly different from each other but were considerably different when compared with the raw material powder. All fine particle fractions attained with the porous particles of sulfadimidine were significantly greater (33.7±3.9% and 41.1±2.1% for the system shown in Example 11 and 12, respectively) than the fine particle fraction measured for either the micronised, crystalline sulfadimidine (2.3±0.7%) or non-porous sulfadimidine (21.9±1.6%). FIG. 16 presents the results graphically. The mass median aerodynamic diameters (MMADs) were also calculated and were 14.4±4.9 μm, 3.4±0.3 μm, 2.9±0.2 μm and 2.5±0.1 μm for the starting powder, non-porous spray dried particles, NPMPs from Example 11 and NPMPs from Example 12 systems, respectively.

Aerosolisation properties of various sufadimidine/lactose carrier blends were also investigated using an Andersen Cascade Impactor. The following systems were investigated:
Micronised sulfadimidine
Non-porous sulfadimidine spray dried from 90% v/v ethanol in the closed mode (powder consisted of smooth, spherical, non-porous particles)
Non-porous sulfadimidine/lactose carrier blend mixed in the ratio 35:65 w/w
Non-porous sulfadimidine/lactose carrier blend mixed in the ratio 1:67.5 w/w
Sulfadimidine NPMPs (spray dried using conditions as outlined in Example 12)
Sulfadimidine NPMPs (spray dried using conditions as outlined in Example 12)/lactose carrier blend mixed in the ratio 35:65 w/w
Sulfadimidine NPMPs (spray dried using conditions as outlined in Example 12)/lactose carrier blend mixed in the ratio 1:67.5 w/w

The fine particle fractions obtained from each of the powders listed above were determined to be following: 1.4±0.1%, 25.4±6.7%, 30.3±2.3%, 46.0±4.7%, 44.7±3.8%, 39.9±2.3% and 47.3±9.1% for micronised sulfadimidine, non-porous spray dried drug, the blend of non-porous sulfadimidine and lactose carrier in the ratio 35:65 w/w, the blend of non-porous sulfadimidine and lactose carrier in the ratio 1:67.5 w/w, sulfadimidine NPMPs, the blend of sulfadimidine NPMPs and lactose carrier in the ratio 35:65 w/w and the blend of sulfadimidine NPMPs and lactose carrier in the ratio 1:67.5 w/w, respectively.

### Example 13

NPMPs from Example 12 were selected for formulation as a suspension and subsequent stability analysis. The flocculation tendency of these NPMPs was compared to that of both raw and non-porous drug (prepared as outlined in Example 12 but with the spray dryer set to the closed mode). The suspension formulations were prepared as described in the Experiemental Section.

Initially all suspensions were of a cloudy, white colour. The powder particles of the raw drug were seen to settle quickly and completely settled within 30 min. The suspension formulated from non-porous particles did not settle as quickly as the raw material. No powder material was floating on top of the suspension. After 4 hours the majority of the particles had sedimented to the bottom of the container. In the suspension of the NPMPs, it was observed after a period of four hours that while some of the particles had settled to the bottom of the graduated cylinder and some were floating at the top of the suspension, a large proportion of the porous particles remained in suspension. After observing the suspensions for 4 hours, it was apparent that the stability of the NPMPs in suspension was superior to that of either the raw material or the smooth spherical particles of the non-porous material.

### Example 14

Solubility studies of NPMPs of sulfadimidine prepared as outlined in Example 12, non-porous drug (prepared as outlined in Example 12 but with the spray dryer set to the closed mode) and starting material were carried out and the results for the sealed ampoule method (static method) and overhead stirrer method (dynamic method) are presented in Tables 2 and 3, respectively.

TABLE 2
Saturated solubility results (sealed ampoule method)
of raw material, spray dried non-porous and NPMPs
of sulfadimidine, after 24 hrs at 37° C.

| | Apparent solubility in water | Apparent solubility in water |
|---|---|---|
| | (mg/ml) | with 1% w/v PVP (mg/ml) |
| Raw Material | 0.672 ± 0.037 | 0.725 ± 0.007 |
| Non Porous | 0.704 ± 0.010 | 0.808 ± 0.024 |
| Porous | 0.755 ± 0.012 | 2.331 ± 0.024 |

TABLE 3
Dynamic solubility results of raw material, spray dried non
porous and NPMPs of sulfadimidine, after 24 hrs at 37° C.

| | Apparent solubility in water | Apparent solubility in water |
|---|---|---|
| | (mg/ml) | with 1% w/v PVP (mg/ml) |
| Raw Material | 0.620 ± 0.01 | 0.745 ± 0.012 |
| Non Porous | 0.606 ± 0.001 | 0.820 ± 0.033 |
| Porous | 0.646 ± 0.01 | 1.427 ± 0.012 |

Sealed ampoule solubility studies in water for the NPMPs indicated a significant increase in solubility in comparison to the raw material. The same method used for the NPMPs in water containing 1% w/v PVP indicated a 3-fold increase in solubility in comparison to the pure crystalline drug, PVP being included in the medium to retard phase transformation of the spray dried material. In water and water containing 1% w/v PVP, recrystallisation of the amorphous phase of the NPMPs occurred completely, as confirmed by XRD and DSC analysis. DSC analysis of SD post 24 hrs in water confirmed one endothermic peak, with an onset of melting at 196.8° C. DSC analysis of sulfadimidine NPMP material post 24 hrs in water containing 1% w/v PVP presented one endothermic peak, with an onset of melting at 196.6° C.

Dynamic solubility studies confirmed that NPMPs have a significant increase in solubility in comparison to the raw crystalline material, and to a lesser extent in comparison to the non-porous material. Dynamic solubility studies of NPMPs in water containing 1% w/v PVP indicated a 1.9-fold increase in solubility.

Sulfadiazine (A Bioactive)

### Example 15

0.1 g sulfadiazine was dissolved in 100 ml of 90% v/v ethanol. This ethanolic mixture of the drug was heated up to ˜40° C. to improve solubility of the active. The resulting solution was clear and the drug concentration was equal to 0.1% w/v. The solution was spray dried using a Büchi B-290 Mini Spray Dryer working in the suction mode. The drying gas utilised was air.

The process parameters were employed as outlined below:
Inlet temperature: 78° C.
Outlet temperature: 52° C.
Aspirator setting: 100%
Airflow rate: 4 cm (670 Nl/h)
Pump setting: 30% (480 ml/h)

The collected powder consisted of porous, irregularly shaped particles. Individual particles were made of fused, but distinguishable spherical particles being 100-200 nm in size. The particles had rough surfaces and XRD analysis showed that the powder was crystalline and the degree of crystallinity was similar to that of the starting material. The SEM micrograph is shown in FIG. 17.

Sulfamerazine (A Bioactive)

### Example 16

0.3 g sulfamerazine was dissolved in 100 ml of 90% v/v ethanol. The drug concentration in the solution was equal to 0.2% w/v. The solution was spray dried using a Büchi B-290 Mini Spray Dryer working in the suction mode. The drying gas utilised was nitrogen.

The process parameters employed were as outlined below:
Inlet temperature: 90° C.
Outlet temperature: 58° C.
Aspirator setting: 100%
Airflow rate: 4 cm (670 Nl/h)
Pump setting: 30% (480 ml/h)

The collected powder constituted of porous, irregularly shaped particles. The particles had rough surfaces. XRD analysis revealed that the powder was crystalline in nature, but the degree of crystallinity was lower than that of the starting material. The SEM micrograph is shown in FIG. 18.

FIG. 15b displays the surface area and bulk density results measured for the raw sulfamerazine powder, non-porous drug (prepared as outlined in Example 16 but the spray dryer set to the closed mode) and NPMPs produced as per Example 16.

For the NPMPs the surface area measured 23.13±0.29 m²/g and bulk density was 0.067±0.007 g/cm³. Another batch of sulfamerazine NPMPs was produced at the lower inlet temperature of 78° C. for which a surface area of 19.70±0.33 m²/g was measured with a bulk density of 0.059±0.005 g/cm³

The respirable fractions of the NPMPs were measured with the use of an Andersen cascade impactor. The fine particle fractions of porous and non-porous sulfamerazine were found to be statistically significantly different and were determined to be 43.6±1.8% and 37.9±1.6%, respectively. FIG. 19 presents the results graphically. The MMAD of porous and non-porous sulfamerazine measured 4.15±0.19 μm and 4.65±0.16 μm respectively, non-porous sulfamerazine measuring a slightly larger MMAD.

Generally, nanoporous microparticles of sulfamerazine were obtained with a Büchi B-290 Mini Spray Dryer working in the suction mode with compressed nitrogen or air when the following conditions were utilised:
90% v/v ethanol
0% and 10% ammonium carbonate (by total weight of dissolved solids)
0.2% and 0.3% w/v concentration of sulfamerazine in the feed solution
78 and 85° C. inlet temperature
100% aspirator setting
670 Nl/h drying medium throughput
30% pump setting
   and
80% v/v ethanol
0% and 10% ammonium carbonate (by total weight of dissolved solids)
0.2% w/v concentration of sulfamerazine in the feed solution
78° C. and 85° C. inlet temperature
100% aspirator setting
670 Nl/h drying medium throughput
30% pump setting

### Example 17

0.4 g sulfamerazine was dissolved in 100 ml of 80% v/v methanol. The drug concentration in the solution was equal to 0.4% w/v. The solution was spray dried using a Büchi B-290 Mini Spray Dryer working in the closed mode. The drying gas utilised was nitrogen.

The process parameters were employed as outlined below:
Inlet temperature: 70° C.
Outlet temperature: 49° C.
Aspirator setting: 100%
Airflow rate: 4 cm (670 Nl/h)
Pump setting: 30% (480 ml/h)

The collected powder constituted of porous, irregularly shaped particles. The particles had rough surfaces. The SEM micrograph is shown in FIG. 20.

### Example 18

Solubility studies of NPMPs of sulfamerazine prepared as outlined in Example 16, non-porous drug (prepared as outlined in Example 16 but with the spray dryer set to the closed mode) and starting material were carried out and the results for the sealed ampoule method (static method) and overhead stirrer method (dynamic method) are presented in Table 4 and 5, respectively.

TABLE 4
Saturated solubility results (sealed ampoule method)
of raw material, spray dried non-porous and NPMPs
of sulfamerazine, after 24 hrs at 37° C.

| | Apparent solubility in water | Apparent solubility in water |
|---|---|---|
| | (mg/ml) | with 1% w/v PVP (mg/ml) |
| Raw Material | 0.201 ± 0.019 | 0.319 ± 0.001 |
| Non Porous | 0.363 ± 0.003 | 0.605 ± 0.002 |
| Porous | 0.350 ± 0.012 | 0.455 ± 0.002 |

TABLE 5
Dynamic solubility results of raw material, spray dried non
porous and NPMPs of sulfamerazine, after 24 hrs at 37° C.

| | Apparent solubility in water | Apparent solubility in water |
|---|---|---|
| | (mg/ml) | with 1% w/v PVP (mg/ml) |
| Raw Material | 0.305 ± 0.016 | 0.283 ± 0.013 |
| Non Porous | 0.417 ± 0.070 | 0.563 ± 0.031 |
| Porous | 0.335 ± 0.016 | 0.561 ± 0.006 |

For the sealed ampoule method, in water porous and non-porous sulfamerazine were converted into polymorph II, as confirmed by XRD and DSC analysis. The crystalline raw material remained in the form of polymorph I. The DSC trace of porous drug post 24 hrs in water indicated the presence of polymorph II. NPMPs in water containing 1% w/v PVP remained in the form of polymorph I, measuring a 1.4-fold increase in solubility when compared to the raw material. Non-porous drug in 1% w/v PVP medium also remained in the form of polymorph I, however having recrystallised to a lesser extent when compared to the porous sample a higher solubility was measured. In the dynamic solubility studies in water, porous and non-porous drug was converted into polymorph II, as confirmed by XRD. Porous sulfamerazine in water containing 1% w/v PVP remained in the form of polymorph I, measuring a 2-fold increase in solubility when compared to the raw material. Non-porous sulfamerazine also remained in the form of polymorph I, however there was no significant difference in solubility between the porous and non-porous material after 24 hrs.

Sodium Cromoglycate (A Bioactive)

### Example 19

0.15 g sodium cromoglycate was dissolved in 32 ml of 1:15 (by volume) water:methanol mixture, and then 30 ml of n-butyl acetate was added to the solution so the final ratio of water, methanol and n-butyl acetate was 1:15:15 (by volume). The drug concentration was equal to 0.24% w/v. The mixture was spray dried using a Büchi B-290 Mini Spray Dryer working in the closed mode with a high efficiency cyclone fitted. The drying gas utilised was nitrogen.

The process parameters were employed as outlined below:
Inlet temperature: 100° C.
Outlet temperature: 60° C.
Aspirator setting: 100%
Airflow rate: 4 cm (670 Nl/h)
Pump setting: 30% (480 ml/h)

The spray dried particles were spherical in morphology, ranging in size from 1-3 μm as observed from SEM micrographs (FIG. 21). Sodium cromoglycate exhibited an amorphous nature after spray drying in comparison to the crystalline raw material.

The bulk and tap densities of the powder were calculated to be 0.114±0.006 g/cm³ and 0.248±0.014 g/cm³, respectively compared to the sodium cromoglycate starting material powder for which the bulk and tap densities were determined to be 0.3411±0.024 g/cm³ and 0.661±0.023 g/cm³.

### Example 20

0.15 g sodium cromoglycate was dissolved in 47.5 ml of methanol, and then 2.5 ml of n-butyl acetate was added to the solution so the final ratio of methanol and n-butyl acetate was 95:5 (by volume). The drug concentration was equal to 0.3% w/v. The mixture was spray dried using a Büchi B-290 Mini Spray Dryer working in the closed mode. The drying gas utilised was nitrogen.

The process parameters were employed as outlined below:
Inlet temperature: 100° C.
Outlet temperature: 60° C.
Aspirator setting: 100%
Airflow rate: 4 cm (670 Nl/h)
Pump setting: 30% (480 ml/h)

The morphology of the obtained particle was different to those described in Example 19. The particles were collapsed and irregular in shape. They consisted of tightly fused nanospherical formations as shown in FIG. 22.

The bulk and tap densities of the powder were calculated to be 0.120±0.004 g/cm³ and 0.231±0.011 g/cm³, respectively.

The respirable fractions of sodium cromoglycate NPMPs produced according to the conditions outlined in Example 19 and 20, measured with the use of an Andersen cascade impactor, were considerably different when compared with the Intal Spincaps® commercial product or the non-porous spray dried drug (processed from a 1% w/v aqueous solution at the inlet temperature of 130° C. in the open mode with air). The fine particle fractions attained with the porous particles of the drug from Example 19 and 20 were significantly greater (53.7±7.5% and 40.3±0.7%, respectively) than the FPF acquired with the Intal formulation (28.1±3.7%). The FPF for the non-porous drug was determined to be 28.1±1.5% which is once again statistically different to the FPFs obtained with NPMPs. FIG. 23 presents the results graphically.

The mass median aerodynamic diameters (MMADs) were also calculated and were 7.6±1.3 μm, 8.0±0.8 μm, 5.0±0.3 μm and 4.1±0.5 μm for the Intal Spincaps formulation, non-porous system and NPMPs produced as per Example 19 and 20, respectively.

Additionally, nanoporous microparticles of sodium cromoglycate were obtained with a Büchi B-290 Mini Spray Dryer working in the closed mode with compressed nitrogen when the following conditions were utilised:
9:1, 8:2 and 7:3 (by volume) mixture of methanol and n-butyl acetate
0.3% w/v concentration of sodium cromoglycate in the feed solution
100° C. inlet temperature
100% aspirator setting
670 Nl/h drying medium throughput
30% pump setting

Betamethasone Base (A Steroid)

### Example 21

0.2 g betamethasone base was dissolved in 50 ml of 90% v/v ethanol. The drug concentration in the solution was equal to 0.4% w/v. The solution was spray dried using a Büchi B-290 Mini Spray Dryer working in the suction mode. The drying gas utilised was nitrogen.

The process parameters were employed as outlined below:
Inlet temperature: 78° C.
Outlet temperature: 48° C.
Aspirator setting: 100%
Airflow rate: 4 cm (670 Nl/h)
Pump setting: 30% (480 ml/h)

These NPMPs varied in size from 0.5-4 μm as evident from the SEM micrograph shown in FIG. 24. The morphology of NPMPs of betamethasone was quite similar to that of NPMPs of budesonide (described in Example 1), particles appearing as spherical formations, consisting of fused nanoparticulate structures of spherical shape, the surfaces of particles being highly irregular with visible holes. The XRD scan for NPMPs of betamethasone was characteristic of a disordered state, showing an amorphous halo pattern in comparison to the crystalline raw material. TGA analysis confirmed a weight loss of 3.0% over the temperature range of 25-100° C. DSC of the NPMPs revealed an exothermic peak at ˜143° C. followed by a melting endotherm at 243° C. in contrast to the starting material for which only a melting peak at 246° C. was detected.

Betamethasone Valerate (A Steroid)

### Example 22

0.5 g betamethasone valerate was dissolved in 100 ml of 90% v/v ethanol. The drug concentration in the solution was equal to 0.5% w/v. The solution was spray dried using a Büchi B-290 Mini Spray Dryer working in the suction mode. The drying gas utilised was air.

The process parameters were employed as outlined below:
Inlet temperature: 78° C.
Outlet temperature: 45-49° C.
Aspirator setting: 100%
Airflow rate: 4 cm (670 Nl/h)
Pump setting: 30% (480 ml/h)

The morphology of the NPMPs produced was comparable to that of NPMPs of budesonide from Example 1 and betamethasone base from Example 21. A sample SEM micrograph is shown in FIG. 25.

Also, nanoporous microparticles of betamethasone valerate were obtained with a Büchi B-290 Mini Spray Dryer working in the suction mode with compressed air when the following conditions were utilised:
80% v/v ethanol
0 and 25% ammonium carbonate (by total weight of dissolved solids)
0.5% w/v concentration of the feed solution
85° C. inlet temperature
100% aspirator setting
670 Nl/h drying medium throughput
30% pump setting

Additionally, nanoporous microparticles of betamethasone valerate were obtained with a Büchi B-290 Mini Spray Dryer working in the closed mode with compressed nitrogen when the following conditions were utilised:
60% v/v ethanol
25% ammonium carbonate (by total weight of dissolved solids)
0.5% w/v concentration of the feed solution
100° C. inlet temperature
100% aspirator setting
670 Nl/h drying medium throughput
30% pump setting
   and
80% v/v methanol
25% ammonium carbonate (by total weight of dissolved solids)
0.5% w/v concentration of the feed solution
100° C. inlet temperature
100% aspirator setting
670 Nl/h drying medium throughput
30% pump setting

Para-Aminosalicyclic Acid (PASA) (A Bioactive)

### Example 23

3 g PASA was dissolved in 100 ml of 95% v/v ethanol. The drug concentration in the solution was equal to 3% w/v. The solution was spray dried using a Büchi B-290 Mini Spray Dryer working in the suction mode. The drying gas utilised was air.

The process parameters were employed as outlined below:
Inlet temperature: 78° C.
Outlet temperature: 51° C.
Aspirator setting: 100%
Airflow rate: 4 cm (670 Nl/h)
Pump setting: 20% (320 ml/h)

The powder produced was crystalline by XRD and DSC and consisted of a mixture of particles which were spherical and porous in nature as well as irregular, rough and non-porous. A sample SEM micrograph is shown in FIG. 26.

A mixture of porous and non-porous particles was also obtained with a Büchi B-290 Mini Spray Dryer working in the suction mode with air when the following conditions were used:
90% v/v ethanol
3 and 4% w/v concentration of the feed solution
78° C. inlet temperature
100% aspirator setting
670 Nl/h drying medium throughput
20% pump setting

### Example 24

2.4 g PASA was dissolved in 100 ml of 90% v/v ethanol and then 0.6 g ammonium carbonate (which constituted 20% by weight of solids) was added to the clear solution of PASA and mixed using a magnetic stirrer until the powder had completely dissolved. The total weight of solids dissolved was 3 g, which gave a solution concentration equal to 3% w/v. The solution was spray dried using a Büchi B-290 Mini Spray Dryer working in the suction mode. The drying gas utilised was air. The process parameters are outlined below:
Inlet temperature: 78° C.
Outlet temperature: 44° C.
Aspirator setting: 100%
Airflow rate: 4 cm (670 Nl/h)
Pump setting: 20% (320 ml/h)

The resulting product consisted of porous particles that were crystalline by XRD. DSC analysis showed a multiple endothermic peak with an onset at ˜130° C. in contrast to a single melting endotherm of the starting material beginning at ˜140° C. No exothermic peak was detected confirming the crystalline property of the spray dried material. The median particle size was ˜3 μm with the particle size distribution being principally monomodal with a small “bump” of the submicron sized particles. The particles were spherical with very rough surfaces. The holes were apparent as fissures on the surface resembling fused nanocrystalline formations. The SEM micrograph is presented in FIG. 27. The bulk and tap densities of were calculated to be 0.12 g/cm³ and 0.17 g/cm³, respectively.

Similarly, NPMPs particles of PASA were also obtained with a Büchi B-290 Mini Spray Dryer working in the suction mode with air when the following conditions were used:
90% v/v ethanol
4% w/v concentration of the feed solution
78° C. inlet temperature
100% aspirator setting
670 Nl/h drying medium throughput
30% pump setting

### Example 25

0.8 g PASA was dissolved in 100 ml of 80% v/v methanol and then 0.2 g ammonium carbonate (which constituted 20% by weight of solids) was added to the solution of PASA and mixed using a magnetic stirrer until a clear solution was obtained. The total weight of solids dissolved was 1 g, which gave a solution concentration equal to 1% w/v. The solution was spray dried using a Büchi B-290 Mini Spray Dryer working in the closed mode. The drying gas utilised was nitrogen.

The process parameters are outlined below:
Inlet temperature: 78° C.
Outlet temperature: 50° C.
Aspirator setting: 100%
Airflow rate: 4 cm (670 Nl/h)
Pump setting: 20% (320 ml/h)

The collected powder exhibited very similar physicochemical properties in terms of XRD and DSC results as the powder described in Example 24. The particles were viewed using SEM (FIG. 28) which revealed spherical morphologies and rough surfaces of the majority of the particles. There were cracks visible on the surfaces. The median particle size was determined to be ˜4 μm, the particle size was monomodal with two minor bumps, one in the submicron sizes and the second between 30 and 100 μm.

Lysozyme (A Protein)

### Example 26

0.225 g lysozyme and 0.025 g ammonium carbonate (which constituted 10% by weight of solids) were dissolved in 10 ml of deionised water, and then ethanol was added to the solution so the final concentration of ethanol was 80% v/v. The total weight of solids was 0.25 g, which gave a concentration equal to 0.5% w/v. The solution was spray dried using a Büchi B-290 Mini Spray Dryer working in the suction mode with compressed nitrogen.

The process parameters are outlined below:
Inlet temperature: 78° C.
Outlet temperature: 49° C.
Aspirator setting: 100%
Airflow rate: 4 cm (670 Nl/h)
Pump setting: 30% (480 ml/h)

The collected powder consisted of spherical particles having evidently porous exteriors. The powder was viewed under SEM and the micrograph is shown in FIG. 29. The median particle size was measured to be 1.4 μm with around 17% (by volume) of particles in the submicron size.

Generally, nanoporous microparticles of lysozyme were obtained with a Büchi B-290 Mini Spray Dryer working in the suction open mode with compressed nitrogen or air when the following conditions were utilised:
70% v/v ethanol
10% ammonium carbonate (by total weight of dissolved solids)
0.5% w/v concentration of lysozyme in the feed solution
78 and 90° C. inlet temperature
100% aspirator setting
670 Nl/h drying medium throughput
30% pump setting
   and
75% v/v ethanol
10% ammonium carbonate (by total weight of dissolved solids)
0.3 and 0.5% w/v concentration of lysozyme in the feed solution
78° C. inlet temperature
100% aspirator setting
670 Nl/h drying medium throughput
30% pump setting
   and
80% v/v ethanol
5% and 10% ammonium carbonate (by total weight of dissolved solids)
0.3%, 0.4% and 0.5% w/v concentration of lysozyme in the feed solution
78° C. and 85° C. inlet temperature
100% aspirator setting
670 Nl/h drying medium throughput
30% pump setting
   and
80% v/v ethanol
10% ammonium hydrogen carbonate (by total weight of dissolved solids)
0.5% w/v concentration of lysozyme in the feed solution
78° C. inlet temperature
100% aspirator setting
670 Nl/h drying medium throughput
30% pump setting
   and
80% v/v ethanol
10% ammonium hydrogen carbonate (by total weight of dissolved solids)
0.5% w/v concentration of lysozyme in the feed solution
78° C. inlet temperature
100% aspirator setting
670 Nl/h drying medium throughput
30% pump setting
   and
80% v/v ethanol
40, 50 and 60% ammonium formate (by total weight of dissolved solids)
0.5% w/v concentration of lysozyme in the feed solution
90° C. inlet temperature
100% aspirator setting
670 Nl/h drying medium throughput
30% pump setting
   and
80% v/v ethanol
50 and 60% ammonium acetate (by total weight of dissolved solids)
0.5% w/v concentration of lysozyme in the feed solution
90° C. inlet temperature
100% aspirator setting
670 Nl/h drying medium throughput
30% pump setting

### Example 27

0.12 g lysozyme and 0.08 g ammonium carbonate (which constituted 40% by weight of solids) were dissolved in 10 ml of deionised water, and then 40 ml methanol was added so the final concentration of methanol was 80% v/v. The total weight of solids was 0.2 g, which gave a concentration equal to 0.4% w/v. Spray drying was performed using a Büchi B-290 Mini Spray Dryer working in the open suction mode with compressed nitrogen.

The process parameters are outlined below:
Inlet temperature: 90° C.
Outlet temperature: 56° C.
Aspirator setting: 100%
Airflow rate: 4 cm (670 Nl/h)
Pump setting: 30% (480 ml/h)

The collected powder consisted of evidently porous, spherical particles and a sample SEM micrograph is shown in FIG. 30.

Additionally, nanoporous microparticles of lysozyme were obtained with a Büchi B-290 Mini Spray Dryer working in the closed mode with compressed nitrogen when the following conditions were utilised:
65, 70 and 75% v/v methanol
40% ammonium carbonate (by total weight of dissolved solids)
0.4% w/v concentration of the feed solution
90° C. inlet temperature
100% aspirator setting
670 Nl/h drying medium throughput
30% pump setting

Trypsin (A Protein)

### Example 28

0.15 g trypsin and 0.1 g ammonium carbonate (which constituted 40% by weight of solids) were dissolved in 2.5 ml of deionised water, and then 47.5 ml ethanol was added so the final concentration of ethanol was 95% v/v. The total weight of solids was 0.25 g, which gave a concentration equal to 0.5% w/v. Spray drying was performed using a Büchi B-290 Mini Spray Dryer working in the open suction mode with compressed air.

The process parameters are outlined below:
Inlet temperature: 78° C.
Outlet temperature: 47° C.
Aspirator setting: 100%
Airflow rate: 4 cm (670 Nl/h)
Pump setting: 30% (480 ml/h)

The spray dried powder consisted of a mixture of evidently porous as well as collapsed, non-porous particles. The SEM micrograph is presented in FIG. 31.

Budesonide/Formoterol Fumarate Dehydrate (Bioactive Combination)

### Example 29

0.25 g budesonide and 0.015 g formoterol fumarate dihydrate was dissolved in 26.5 ml of 80% v/v ethanol. The drug concentration in the solution was equal to 1% w/v. The solution was spray dried using a Büchi B-290 Mini Spray Dryer working in the suction mode. The drying gas utilised was air.

The process parameters were employed as outlined below:
Inlet temperature: 78° C.
Outlet temperature: 48° C.
Aspirator setting: 100%
Airflow rate: 4 cm (670 Nl/h)
Pump setting: 20% (320 ml/h)

The outer morphology of these particles resembled those of budesonide spray dried as outlined in Example 1 and 2. A sample SEM micrograph is presented in FIG. 32.

### Bendroflumethiazide/Sulfadimidine (Bioactive Combination)

### Example 30

0.25 g bendroflumethiazide and 0.25 g sulfadimidine was dissolved in 50 ml of 80% v/v ethanol. The drug concentration in the solution was equal to 1% w/v. The solution was spray dried using a Büchi B-290 Mini Spray Dryer working in the suction mode. The drying gas utilised was air.

The process parameters were employed as outlined below:
Inlet temperature: 78° C.
Outlet temperature: 47° C.
Aspirator setting: 100%
Airflow rate: 4 cm (670 Nl/h)
Pump setting: 20% (320 ml/h)

The outer morphology of these particles resembled those of bendroflumethiazide spray dried as outlined in Example 5 and 7. A sample SEM micrograph is presented in FIG. 33.

Trehalose (An Excipient)

### Example 31

0.25 g trehalose dihydrate was dissolved in 40 ml of methanol, and then 10 ml of n-butyl acetate was added to the solution so the final ratio of methanol and n-butyl acetate was 8:2 (by volume). The sugar concentration in the solution was equal to 0.5% w/v. The solution was spray dried using a Büchi B-290 Mini Spray Dryer working in the closed mode. The drying gas utilised was nitrogen.

The process parameters were employed as outlined below:
Inlet temperature: 100° C.
Outlet temperature: 65° C.
Aspirator setting: 100%
Airflow rate: 4 cm (670 Nl/h)
Pump setting: 30% (480 ml/h)

The spray dried powder constituted of spheres of nanoporous microparticles. A sample SEM micrograph is presented in FIG. 34. Product with a similar morphology was also obtained with a high efficiency cyclone fitted to the spray dryer.
Additionally, nanoporous microparticles of trehalose were obtained with a Büchi B-290 Mini Spray Dryer working in the closed mode with compressed nitrogen when the following conditions were utilised:
1:1 mixture of methanol and n-butyl acetate
0.5% w/v concentration of the feed solution
100° C. inlet temperature
100% aspirator setting
670 Nl/h drying medium throughput
30% pump setting

Raffinose (An Excipient)

### Example 32

0.5 g raffinose pentahydrate was dissolved in 40 ml of methanol, and then 10 ml of n-butyl acetate was added to the solution so the final ratio of methanol and n-butyl acetate was 8:2 (by volume). The sugar concentration in the solution was equal to 1% w/v. The solution was spray dried using a Büchi B-290 Mini Spray Dryer working in the closed mode. The drying gas utilised was nitrogen.

The process parameters were employed as outlined below:
Inlet temperature: 100° C.
Outlet temperature: 63° C.
Aspirator setting: 100%
Airflow rate: 4 cm (670 Nl/h)
Pump setting: 30% (480 ml/h)

The spray dried powder constituted of spheres of nanoporous microparticles. A sample SEM micrograph is presented in FIG. 35.

Additionally, a mixture of NPMPs and non-porous particles of raffinose were obtained with a Büchi B-290 Mini Spray Dryer working in the closed mode with compressed nitrogen when the following conditions were utilised:
1:1 mixture of methanol and n-butyl acetate
1% w/v concentration of the feed solution
100° C. inlet temperature
100% aspirator setting
670 Nl/h drying medium throughput
30% pump setting

Hydroxypropyl-β-Cyclodextrin (HPBCD) (An Excipient)

### Example 33

0.6 g HPBCD was dissolved in 32.5 ml of 1:6:6 (by volume) mixture of water, methanol and n-butyl acetate: The polymer concentration in the solution was equal to 1.8% w/v. The solution was spray dried using a Büchi B-290 Mini Spray Dryer working in the closed mode. The drying gas utilised was nitrogen.

The process parameters were employed as outlined below:
Inlet temperature: 100° C.
Outlet temperature: 65° C.
Aspirator setting: 100%
Airflow rate: 4 cm (670 Nl/h)
Pump setting: 30% (480 ml/h)

The spray dried powder constituted of slightly deformed porous spheres. A sample SEM micrograph is presented in FIG. 36.

Additionally, nanoporous microparticles of HPBCD were obtained with a Büchi B-290 Mini Spray Dryer working in the closed mode with compressed nitrogen when the following conditions were utilised:
1:15:15 mixture of water, methanol and n-butyl acetate
1.9% w/v concentration of HPBCD in the feed solution
100° C. inlet temperature
100% aspirator setting
670 Nl/h drying medium throughput
30% pump setting

### Example 34

5 g HPBCD was dissolved in 250 ml of 1:1 (by volume) mixture of methanol and n-butyl acetate. The polymer concentration in the solution was equal to 2% w/v. The solution was spray dried using a Büchi B-290 Mini Spray Dryer working in the closed mode. The drying gas utilised was nitrogen.

The process parameters were employed as outlined below:
Inlet temperature: 100° C.
Outlet temperature: 66° C.
Aspirator setting: 100%
Airflow rate: 4 cm (670 Nl/h)
Pump setting: 30% (480 ml/h)

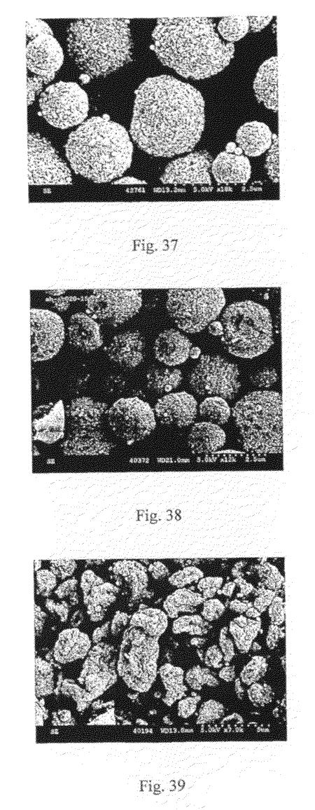

SEM (FIG. 37) analysis showed that the resulting powder consisted of spherical nanoporous particles.

### Example 35

0.6 g HPBCD was dissolved in 60 ml of 1:1 (by volume) mixture of methanol and n-propyl acetate. The polymer concentration in the solution was equal to 1% w/v. The solution was spray dried using a Büchi B-290 Mini Spray Dryer working in the closed mode. The drying gas utilised was nitrogen.

The process parameters were employed as outlined below:
Inlet temperature: 100° C.
Outlet temperature: 66° C.
Aspirator setting: 100%
Airflow rate: 4 cm (670 Nl/h)
Pump setting: 30% (480 ml/h)

SEM (FIG. 38) analysis showed that the resulting powder consisted of spherical nanoporous particles.

Additionally, nanoporous microparticles of HPBCD were obtained with a Büchi B-290 Mini Spray Dryer working in the closed mode with compressed nitrogen when the following conditions were utilised:
1:1 mixture of methanol and n-propyl acetate
2% and 4% w/v concentration of HPBCD in the feed solution
85° C., 100° C. and 120° C. inlet temperature
100% aspirator setting
670 Nl/h drying medium throughput
30% pump setting
   and
3:2 mixture of methanol and n-propyl acetate
2.4% w/v concentration of HPBCD in the feed solution
100° C. inlet temperature
100% aspirator setting
670 Nl/h drying medium throughput
30% pump setting

### Example 36

0.6 g HPBCD was dissolved in 60 ml of 1:1 (by volume) mixture of methanol and isopropyl acetate. The polymer concentration in the solution was equal to 1% w/v. The solution was spray dried using a Büchi B-290 Mini Spray Dryer working in the closed mode. The drying gas utilised was nitrogen.

The process parameters were employed as outlined below:
Inlet temperature: 100° C.
Outlet temperature: 61-63° C.
Aspirator setting: 100%
Airflow rate: 4 cm (670 Nl/h)
Pump setting: 30% (480 ml/h)

The spray dried particles were evidently porous in nature (as seen by SEM presented in FIG. 39) but their shapes were distorted and irregular.

Additionally, nanoporous microparticles of HPBCD were obtained with a Büchi B-290 Mini Spray Dryer working in the closed mode with compressed nitrogen when the following conditions were utilised:
1:1 mixture of methanol and isopropyl acetate
2% w/v concentration of HPBCD in the feed solution
85° C., 100° C. and 120° C. inlet temperature
100% aspirator setting
670 Nl/h drying medium throughput
15% and 30% pump setting

Polyvinylpyrrolidone 10,000 (PVP 10,000) (An Excipient)

### Example 37

2.4 g PVP 10,000 was dissolved in 120 ml of 1:1 (by volume) mixture of methanol and n-butyl acetate. The polymer concentration in the solution was equal to 2% w/v. The solution was spray dried using a Büchi B-290 Mini Spray Dryer working in the closed mode. The drying gas utilised was nitrogen.

The process parameters were employed as outlined below:
Inlet temperature: 100° C.
Outlet temperature: 61° C.
Aspirator setting: 100%
Airflow rate: 4 cm (670 Nl/h)
Pump setting: 30% (480 ml/h)

The spray dried powder constituted of spherical, evidently porous particles. A sample SEM micrograph is presented in FIG. 40.

Generally, nanoporous microparticles of PVP 10,000 were obtained with a Büchi B-290 Mini Spray Dryer working in the closed mode with compressed nitrogen when the following conditions were utilised:
1:1 mixture of methanol and n-butyl acetate
1%, 2% and 4% w/v concentration of PVP 10,000 in the feed solution
100° C., 120° C. and 130° C. inlet temperature
100% aspirator setting
670 Nl/h drying medium throughput
30% pump setting
   and
3:2 mixture of methanol and n-butyl acetate
2.4% w/v concentration of PVP 10,000 in the feed solution
120° C. inlet temperature
100% aspirattir setting
670 Nl/h drying medium throughput
30% pump setting
   and
3:1 mixture of methanol and n-butyl acetate
3% w/v concentration of PVP 10,000 in the feed solution
120° C. inlet temperature
100% aspirator setting
670 Nl/h drying medium throughput
30% pump setting
   and
2:3 mixture of methanol and n-butyl acetate
2.4% w/v concentration of PVP 10,000 in the feed solution
120° C. inlet temperature
100% aspirator setting
670 Nl/h drying medium throughput
30% pump setting

Polyvinylpyrrolidone 40,000 (PVP 40,000) (An Excipient)

### Example 38

5 g PVP 40,000 was dissolved in 250 ml of 1:1 (by volume) mixture of methanol and n-butyl acetate. The polymer concentration in the solution was equal to 2% w/v. The solution was spray dried using a Büchi B-290 Mini Spray Dryer working in the closed mode. The drying gas utilised was nitrogen.

The process parameters were employed as outlined below:
Inlet temperature: 100° C.
Outlet temperature: 67° C.
Aspirator setting: 100%
Airflow rate: 4 cm (670 Nl/h)
Pump setting: 30% (480 ml/h)

The spray dried powder constituted of slightly deformed spheres of nanoporous microparticles. A sample SEM micrograph is presented in FIG. 41.

Additionally, nanoporous microparticles of PVP 40,000 were obtained with a Büchi B-290 Mini Spray Dryer working in the closed mode with compressed nitrogen when the following conditions were utilised:
1:1 mixture of methanol and n-butyl acetate
2% w/v concentration of PVP 40,000 in the feed solution
100° C. and 120° C. inlet temperature
100% aspirator setting
670 Nl/h drying medium throughput
30% pump setting

Budesonide/Hydroxypropyl-β-Cyclodextrin (HPBCD) (Bioactive-Excipient Combination)

### Example 39

0.1 g budesonide and 0.5 g HPBCD was dissolved in 30 ml of 1:1 (by volume). mixture of methanol and n-butyl acetate. The concentration of the resulting solution was equal to 2% w/v total solute concentration. The mixture was then spray dried using a Büchi B-290 Mini Spray Dryer working in the closed mode. The drying gas utilised was nitrogen.

The process parameters were employed as outlined below:
Inlet temperature: 100° C.
Outlet temperature: 74° C.
Aspirator setting: 100%
Airflow rate: 4 cm (670 Nl/h)
Pump setting: 30% (480 ml/h)

The spray dried powder constituted of spherical nanoporous microparticles. A sample SEM micrograph is presented in FIG. 42.

Sulfadimidie/Polyvinylpyrrolidone 10,000 (PVP 10,000) (Bioactive-Excipient Combination)

### Example 40

0.81 g sulfadimidine and 0.09 g PVP 10,000 was dissolved in 100 ml of 80% v/v ethanol and then 0.1 g ammonium carbonate (which constituted 10% by weight of solids) was added to the clear solution of the drug and polymer and mixed using a magnetic stirrer until the powder had completely dissolved. The total weight of solids dissolved was 1 g, which gave a solution concentration equal to 1% w/v and PVP constituted 10% (by weight) of the mixture of pharmaceuticals. The solution was spray dried using a Büchi B-290 Mini Spray Dryer working in the suction mode. The drying gas utilised was nitrogen. The process parameters are outlined below:
Inlet temperature: 78° C.
Outlet temperature: 48° C.
Aspirator setting: 100%
Airflow rate: 4 cm (670 Nl/h)
Pump setting: 30% (480 ml/h)

The addition of the hydrophilic polymer PVP resulted in an increase in the solubility of the drug, leading to an increase in the feed concentration and thus yield. As evident in FIG. 43a the morphology of the porous particles was similar to that of porous budesonide (Example 1) and betamethasone base (Example 21), consisting of spherical particles of fused nanoparticulate structures, the surfaces of particles being highly irregular with visible holes. The morphology of these NPMPs is significantly different from that of excipient free NPMPs of sulfadimidine. XRD analysis confirmed an amorphous state.

The exclusion of ammonium carbonate in this system resulted in a retained porous morphology and amorphous state of the particles. Changing the drug:polymer ratio from 9:1 to 8:2 produced a significant effect on the morphology of the particles as evident in FIG. 43b forming irregularly shaped, collapsed, but still porous particles.

Bendroflumethiazide/Polyvinylpyrrolidone 10,000 (PVP 10,000) (Bioactive-Excipient Combination)

### Example 41

1.62 g bendroflumethiazide and 0.18 g PVP 10,000 was dissolved in 100 ml of 80% v/v ethanol and then 0.2 g ammonium carbonate (which constituted 10% by weight of solids) was added to the clear solution of the drug and polymer and mixed using a magnetic stirrer until the powder had completely dissolved. The total weight of solids dissolved was 2 g, which gave a solution concentration equal to 2% w/v and PVP constituted 10% (by weight) of the mixture of pharmaceuticals. The solution was spray dried using a Büchi B-290 Mini Spray Dryer working in the suction mode. The drying gas utilised was nitrogen. The process parameters are outlined below:
Inlet temperature: 78° C.
Outlet temperature: 47° C.
Aspirator setting: 100%
Airflow rate: 4 cm (670 Nl/h)
Pump setting: 30% (480 ml/h)

In this instance both drug/polymer ratios of 9:1 and 1:1 resulted in porous particle production. As evident in FIG. 44a (BFMT/PVP in the ratio 9:1) and FIG. 44b (BFMT/PVP in the ratio 1:1) the particles appear as roughly spherical formations with irregular surfaces consisting of fused/sintered nanoparticulate structures.

Additionally, nanoporous microparticles of bendroflumethiazide/PVP 10,000 were obtained with a Büchi B-290 Mini Spray Dryer working in the closed mode with compressed nitrogen when the following conditions were utilised:
2% w/v concentration of the feed solution
1:1 mixture of methanol and n-butyl acetate
100° C. inlet temperature
100% aspirator setting
670 Nl/h drying medium throughput
30% pump setting

Bendroflumethiazide/Magnesium Stearate (Bioactive-Excipient Combination)

### Example 42

2.2275 g bendroflumethiazide was dissolved in 100 ml of 80% v/v ethanol and then 0.0225 g magnesium stearate was dispersed in the ethanolic solution of the drug. Finally, 0.25 g ammonium carbonate (which constituted 10% by weight of solids) was added to the mixture of bendroflumethiazide and magnesium stearate and mixed using a magnetic stirrer until the powder had completely dissolved. The total weight of solids dissolved was 2.5 g, which gave a solution concentration equal to 2.5% w/v and magnesium stearate constituted 1% (by weight) of the mixture of pharmaceuticals. The solution was spray dried using a Büchi B-290 Mini Spray Dryer working in the suction mode with compressed air.

The process parameters are outlined below:
Inlet temperature: 78° C.
Outlet temperature: 48° C.
Aspirator setting: 100%
Airflow rate: 4 cm (670 Nl/h)
Pump setting: 30% (480 ml/h)

The powder obtained was composed of irregular, sponge-like nanoporous particles and a sample SEM micrograph is presented in FIG. 45.

Additionally, nanoporous microparticles of bendroflumethiazide/magnesium stearate were obtained with a Büchi B-290 Mini Spray Dryer working in the suction mode with compressed nitrogen when the following conditions were utilised:
1% w/v concentration of magnesium stearate in the feed solution
80% v/v ethanol
78° C. inlet temperature
100% aspirator setting
670 Nl/h drying medium throughput
30% pump setting
   and
0.5 and 2% w/v concentration of magnesium stearate in the feed solution
10% ammonium carbonate (by total weight of dissolved solids)
80% v/v ethanol
78° C. inlet temperature
100% aspirator setting
670 Nl/h drying medium throughput
30% pump setting
   and when a Büchi B-290 Mini Spray Dryer working in the closed mode with compressed nitrogen was utilised:
1% w/v concentration of magnesium stearate in the feed solution
10% ammonium carbonate (by total weight of dissolved solids)
80% v/v methanol
70, 90 and 110° C. inlet temperature
100% aspirator setting
670 Nl/h drying medium throughput
30% pump setting

Sulfadimidine/Magnesium Stearate (Bioactive-Excipient Combination

### Example 43

0.2686 g sulfadimidine was dissolved in 100 ml of 80% v/v ethanol and then 0.0013 g magnesium stearate was dispersed in the ethanolic solution of the drug. Finally, 0.03 g ammonium carbonate (which constituted 10% by weight of solids) was added to the mixture of sulfadimidine and magnesium stearate and mixed using a magnetic stirrer until the powder had completely dissolved. The total weight of solids dissolved was 0.3 g, which gave a solution concentration equal to 0.3% w/v and magnesium stearate constituted 0.5% (by weight) of the mixture of pharmaceuticals. The solution was spray dried using a Büchi B-290 Mini Spray Dryer working in the open suction mode with compressed nitrogen.

The process parameters are outlined below:
Inlet temperature: 78° C.
Outlet temperature: 48° C.
Aspirator setting: 100%
Airflow rate: 4 cm (670 Nl/h)
Pump setting: 30% (480 ml/h)

As evident from FIG. 46a the resulting powder consisted of irregularly shaped, deformed porous particles, in comparison to the spherical uniform excipient free NPMPs of sulfadimidine produced as outlined in Example 12. There was evidence of some non-porous deformed particles.

When the content of magnesium stearate was increased to 1% w/v, the process resulted in the formation of some spherical porous particles as evident from FIG. 46b, however there was a number of non-porous spherical particles visible in the sample.

Lysozyme/Hydroxypropyl-β-Cyclodextrin (HPBCD) (Bioactive-Excipient Combination)

### Example 44

0.08 g lysozyme and 0.32 g HPBCD was dissolved in 20 ml of methanol, and then 20 ml of n-butyl acetate was added to the solution so the final ratio of methanol and n-butyl acetate was 1:1 (by volume). The concentration of the resulting dispersion was equal to 1% w/v. The mixture was then spray dried using a Büchi B-290 Mini Spray Dryer working in the closed mode. The drying gas utilised was nitrogen.

The process parameters were employed as outlined below:
Inlet temperature: 100° C.
Outlet temperature: 67° C.
Aspirator setting: 100%
Airflow rate: 4 cm (670 Nl/h)
Pump setting: 30% (480 ml/h)

The spray dried powder constituted of spherical nanoporous microparticles. A sample SEM micrograph is presented in FIG. 47.

Lysozyme/Trehalose (Bioactive-Excipient Combination)

### Example 45

0.2025 g lysozyme, 0.0225 g trehalose dihydrate and 0.025 g ammonium carbonate was dissolved in 15 ml of deionised water, and then 35 ml of ethanol was added to the solution, so the final concentration of ethanol was 70% v/v. The concentration of the resulting dispersion was equal to 1% w/v and the ratio of lysozyme and sugar was 9:1 (by weight). The mixture was then spray dried using a Büchi B-290 Mini Spray Dryer working in the suction mode. The drying gas utilised was nitrogen.

The process parameters were employed as outlined below:
Inlet temperature: 90° C.
Outlet temperature: 54° C.
Aspirator setting: 100%
Airflow rate: 4 cm (670 Nl/h)
Pump setting: 30% (480 ml/h)

The spray dried powder constituted of spherical nanoporous microparticles. A sample SEM micrograph is presented in FIG. 48.

Additionally, nanoporous microparticles of lysozyme/trehalose were obtained with a Büchi B-290 Mini Spray Dryer working in the suction mode with compressed nitrogen or air when the following conditions were utilised:
70% v/v ethanol
8:2, 7:3 and 1:1 ratio (by weight) of lysozyme and trehalose
10% ammonium carbonate (by total weight of dissolved solids)
0.5% w/v concentration of the feed solution
90° C. inlet temperature
100% aspirator setting
670 Nl/h drying medium throughput
30% pump setting
   and
70% v/v ethanol
1:1 ratio (by weight) of lysozyme and trehalose
10% ammonium carbonate (by total weight of dissolved solids)
0.5 and 1% w/v concentration of the feed solution
78 and 90° C. inlet temperature
100% aspirator setting
670 Nl/h drying medium throughput
30% pump setting

Lysozyme/Raffinose (Bioactive-Excipient Combination)

### Example 46

0.225 g lysozyme, 0.225 g raffinose pentahydrate and 0.05 g ammonium carbonate was dissolved in 30 ml of deionised water, and then 70 ml of ethanol was added to the solution, so the final concentration of ethanol was 70% v/v. The concentration of the resulting dispersion was equal to 0.5% w/v and the ratio of lysozyme and sugar was 1:1 (by weight). The mixture was then spray dried using a Büchi B-290 Mini Spray Dryer working in the suction mode. The drying gas utilised was air.

The process parameters were employed as outlined below:
Inlet temperature: 90° C.
Outlet temperature: 51-54° C.
Aspirator setting: 100%
Airflow rate: 4 cm (670 Nl/h)
Pump setting: 30% (480 ml/h)

The spray dried powder constituted of spherical but highly folded porous microparticles. A sample SEM micrograph is presented in FIG. 49.

Hydrochlorothiazide/Polyvinylpyrrolidone 10,000 (PVP 10,000) (Bioactive-Excipient Combination)

### Example 47

2.5 g hydrochlorothiazide and 2.5 g PVP 10,000 was dissolved in 290 ml of 1:1 (by volume) mixture of methanol and n-butyl acetate. The concentration of the resulting solution was equal to 1.72% w/v. The mixture was then spray dried using a Büchi B-290 Mini Spray Dryer working in the closed mode. The drying gas utilised was nitrogen.

The process parameters were employed as outlined below:
Inlet temperature: 100° C.
Outlet temperature: 72° C.
Aspirator setting: 100%
Airflow rate: 4 cm (670 Nl/h)
Pump setting: 30% (480 ml/h)

The spray dried powder constituted of spherical nanoporous microparticles. A sample SEM micrograph is presented in FIG. 50.

This system was subjected to solid state stability studies at both sets of environmental conditions outlined in the Experimental Section. After storing for 38 days at 25° C. and 60% relative humidity the sample was still amorphous and keeping its original porous morphology, whereas porous PVP 10,000 spray dried alone and hydrochlorothiazide system spray dried alone had both lost their original morphologies. However, when kept at 40° C. and 75% relative humidity, the system was not stable and recrystallised.

Bendroflumethiazide/Hydroxypropyl-β-Cyclodextrin (HPBCD) (Bioactive-Excipient Combination)

### Example 48

0.1 g bendroflumethiazide and 0.5 g HPBCD was dissolved in 30 ml of 1:1 (by volume) mixture of methanol and n-butyl acetate. The concentration of the resulting solution was equal to 2% w/v. The mixture was then spray dried using a Büchi B-290 Mini Spray Dryer working in the closed mode. The drying gas utilised was nitrogen.

The process parameters were employed as outlined below:
Inlet temperature: 100° C.
Outlet temperature: 69° C.
Aspirator setting: 100%
Airflow rate: 4 cm (670 Nl/h)
Pump setting: 30% (480 ml/h)

The spray dried powder constituted of compact, spherical nanoporous microparticles. A sample SEM micrograph is presented in FIG. 51.

Bendroflumethiazide/Polyvinylpyrrolidone 40,000 (PVP 40,000) (Bioactive-Excipient Combination)

### Example 49

2.5 g bendroflumethiazide and 2.5 g PVP 40,000 was dissolved in 250 ml of 1:1 (by volume) mixture of methanol and n-butyl acetate. The concentration of the resulting solution was equal to 2% w/v. The mixture was then spray dried using a Büchi B-290 Mini Spray Dryer working in the closed mode. The drying gas utilised was nitrogen.

The process parameters were employed as outlined below:
Inlet temperature: 100° C.
Outlet temperature: 73° C.
Aspirator setting: 100%
Airflow rate: 4 cm (670 Nl/h)
Pump setting: 30% (480 ml/h)

The spray dried powder constituted of compact, spherical nanoporous microparticles. A sample SEM micrograph is presented in FIG. 52.

Bendroflumethiazide/Polyvinylpyrrolidone 1,300,000 (PVP 1300,000) (Bioactive-Excipient Combination)

### Example 50

1.62 g bendroflumethiazide and 0.18 g of PVP 1,300,000 was dissolved in 100 ml of 80% v/v ethanol and then 0.2 g ammonium carbonate (which constituted 10% by weight of solids) was dissolved in the ethanolic solution of the drug. The total weight of solids dissolved was 2 g, which gave a solution concentration equal to 2% w/v. The solution was spray dried using a Büchi B-290 Mini Spray Dryer working in the suction mode with compressed air.

The process parameters were employed as outlined below:
Inlet temperature: 78° C.
Outlet temperature: 48° C.
Aspirator setting: 100%
Airflow rate: 4 cm (670 Nl/h)
Pump setting: 30% (480 ml/h)

The spray dried powder constituted of spherical nanoporous microparticles. A sample SEM micrograph is presented in FIG. 53.

Hydroflumethiazide/Polyvinylpyrrolidone 10,000 (PVP 10,000) (Bioactive-Excipient Combination)

### Example 51

0.3 g hydroflumethiazide and 0.3 g PVP 10,000 was dissolved in 40 ml of 1:1 (by volume) mixture of methanol and n-butyl acetate. The concentration of the resulting solution was equal to 1.5% w/v. The mixture was then spray dried using a Büchi B-290 Mini Spray Dryer working in the closed mode. The drying gas utilised was nitrogen.

The process parameters were employed as outlined below:
Inlet temperature: 100° C.
Outlet temperature: 66° C.
Aspirator setting: 100%
Airflow rate: 4 cm (670 Nl/h)
Pump setting: 30% (480 ml/h)

The spray dried powder constituted of compact, spherical nanoporous microparticles. A sample SEM micrograph is presented in FIG. 54.

Hydrochlorothiazide/Hydroxypropyl-β-Cyclodextrin (HPBCD) (Bioactive-Excipient Combination)

### Example 52

0.3 g hydrochlorothiazide and 0.3 g HPBCD was dissolved in 30 ml of 1:1 (by volume) mixture of methanol and n-butyl acetate. The concentration of the resulting solution was equal to 2% w/v. The mixture was then spray dried using a Büchi B-290 Mini Spray Dryer working in the closed mode. The drying gas utilised was nitrogen.

The process parameters were employed as outlined below:
Inlet temperature: 100° C.
Outlet temperature: 63° C.
Aspirator setting: 100%
Airflow rate: 4 cm (670 Nl/h)
Pump setting: 30% (480 ml/h)

The spray dried powder constituted of spherical nanoporous microparticles. A sample SEM micrograph is presented in FIG. 55.

Hydroxypropyl-β-Cyclodextrin (HPBCD)/Polyvinylpyrrolidone 10,000 (PVP 10,000) (Excipient-Excipient Combination)

### Example 53

0.3 g PVP 10,000 and 0.3 g HPBCD was dissolved in 30 ml of 1:1 (by volume) mixture of methanol and n-butyl acetate. The concentration of the resulting solution was equal to 2% w/v. The mixture was then spray dried using a Büchi B-290 Mini Spray Dryer working in the closed mode. The drying gas utilised was nitrogen.

The process parameters were employed as outlined below:
Inlet temperature: 100° C.
Outlet temperature: 58° C.
Aspirator setting: 100%
Airflow rate: 4 cm (670 Nl/h)
Pump setting: 30% (480 ml/h)

The spray dried powder constituted of compact, spherical nanoporous microparticles. A sample SEM micrograph is presented in FIG. 56.

The invention is not limited to the embodiments hereinbefore described which may be varied in detail.

1-62. (canceled)

63. A method of preparing porous microparticles comprising the steps of:
combining one or more organic compounds with a volatile solvent system; and
drying the system thus formed to provide substantially pure porous microparticles of the organic compound or composite porous microparticles of combinations of organic compounds.

64. The method as claimed in claim 63 wherein the volatile solvent system is a single phase solution.

65. The method as claimed in claim 63 wherein the step of drying the system comprises spray drying, such as spray drying at an inlet temperature of from about 30° C. to about 220° C. or from about 70° C. to about 130° C.

66. The method as claimed in claim 65 wherein the spray drying is carried out in air or in an inert atmosphere, such as nitrogen.

67. The method as claimed in claim 63 wherein the microparticles have an average diameter of between about 0.5 μm to about 10 μm.

68. The method as claimed in claim 63 wherein the microparticles are nanoporous, the microparticles may have an average pore diameter of between about 20 nm to about 1000 nm, the pores may be substantially spherical in shape.

69. The method as claimed in claim 63 wherein the volatile solvent system comprises a mixture of solvents, one of the solvents may be water, the volatile solvent may be an aliphatic hydrocarbon, an aromatic hydrocarbon, a halogenated hydrocarbon, an alcohol, an aldehyde, a ketone, an ester, an ether or mixtures thereof, the solvent system may comprise ethanol and/or methanol.

70. The method as claimed in claim 69 wherein the solvent system comprises from about 5% to about 40% v/v of water such as from about 10% to about 20% v/v of water.

71. The method as claimed in claim 69 wherein the spray drying is carried out at an inlet temperature of from about 70 to about 110° C. for ethanol systems or wherein the spray drying is carried out at an inlet temperature of from about 60° C. to about 130° C. for methanol systems.

72. The method as claimed in claim 63 wherein the system further comprises a process enhancer.

73. The method as claimed in claim 72 wherein the process enhancer is ammonium carbonate.

74. The method as claimed in claim 72 wherein the process enhancer is present in an amount of from about 5% to about 70% by weight of solids such as from about 10% to about 25% by weight of solids.

75. The method as claimed in claim 63 wherein the organic compound is a solid material, the organic compound may be one or more of a bioactive, pharmaceutically acceptable excipient, a pharmaceutically acceptable adjuvant, or combinations thereof, the bioactive may be a protein, peptide or polypeptide.

76. The method as claimed in claim 63 wherein the organic compound is one or more selected from the group comprising: Bendroflumethiazide, Betamethasone base, Betamethasone valerate, Budesonide, Formoterol fumarate, Hydrochlorothiazide, Hydroflumethiazide, Lysozyme, Para-aminosalicylic acid, Sodium cromoglycate, Sulfadiazine, Sulfadimidine, Sulfamerazine, Trypsin, Insulin, Human growth hormone, Somatotropin, Tissue plasminogen activator, Erthyropoietin, Granulocyte colony stimulating factor (G-CSF), Factor VIII, Interferon-α, Interferon-β, IL-2, Calcitonin, Monoclonal antibodies, Therapeutic proteins/peptides/polypeptides, Therapeutic proteins derived from plants, animals, or microorganisms, and recombinant versions of these products, Monoclonal antibodies, Proteins intended for therapeutic use, cytokines, interferons, enzymes, thrombolytics, and other novel proteins, Immunomodulators, Growth factors, cytokines, and monoclonal antibodies intended to mobilize, stimulate, decrease or otherwise alter the production of hematopoietic cells in vivo, magnesium stearate monosaccharides, for example glucose, galactose, fructose and the like; disaccharides, for example trehalose, maltose, lactose, sucrose and the like; trisaccharides, for example raffinose, acarbose, melezitose and the like; cyclic oligosaccharides/cyclodextrins, for example hydroxpropyl-β-cyclodextrin, hydroxyethyl-β-cyclodextrin, α-cyclodextrin, β-cyclodextrin, γ-cyclodextrin, methyl-β-cyclodextrin, dimethyl-β-cyclodextrin, sulfobutylether-β-cyclodextrin, randomly methylated-β-cyclodextrin and the like; soluble polymers, for example polyvinylpyrrolidone such as PVP 10,000, PVP 40,000, PVP 1,300,000, polyethylene glycol and the like; sugar alcohols/polyols, for example mannitol, xylitol, sorbitol and the like; amino sugars and oligosaccharides, for example inulin and maltodextrin and the like; polysaccharides, for example starch, glycogen and the like; cellulose and cellulose derivatives, for example methylcellulose, ethylcellulose, hydroxypropylmethyl cellulose and the like; deoxy, amino and other sugar derivatives, for example deoxy-glucose, deoxy-ribose, galactosamine and the like, alpha and beta adrenoreceptor agonists for example salbutamol, salmeterol, terbutaline, bambuterol, clenbuterol, metaproterenol, fenoterol, rimiterol, reproterol, bitolterol, tulobuterol, isoprenaline, isoproterenol and the like and their salts, anticholinergics for example ipratropium, oxitropium and tiotropium and the like and their salts, glucocorticoids for example beclomethasone, betamethasone, budesonide, ciclesonide, formoterol, fluticasone, mometasone, triamcinolone and the like and their salts and esters, antiallergics for example nedocromil sodium and sodium cromoglycate and the like, leukotriene inhibitors and antagonists for example montelukast, pranlukast, zafirlukast and zileuton and the like, xanthines for example aminophylline, diprophylline, etofylline, proxyphylline, theobromine and theophylline and the like, anti-infectives for example tobramycin, amikacin, ciprofloxacin, gentamicin, para-aminosalicylic acid, rifampicin, isoniazid, capreomycin, acyclovir and ritonavir and the like, antihistamines for example, terfenadine, cetrizine, loratadine and the like, pain control substances for example morphine and codeine and the like and their salts, and combinations thereof.

77. Substantially pure porous microparticles of an organic compound, the microparticles may comprise spherical aggregates of organic compound, the microparticles may comprise sponge-like particles of organic compound, the microparticles may comprise substantially hollow spheres with nanopores in the shell.

78. The porous microparticles as claimed in claim 77 wherein the microparticles are nanoporous.

79. The porous microparticles as claimed in claim 77 wherein the microparticles have an average pore diameter of between about 20 nm to about 1000 nm.

80. The porous microparticles as claimed in claim 77 wherein the pores are substantially spherical in shape.

81. The porous microparticles as claimed in claim 77 which do not contain a surfactant or surfactant residue.

82. The porous microparticles as claimed in claim 77 wherein the organic compound is one or more of a bioactive, pharmaceutically acceptable excipient, a pharmaceutically acceptable adjuvant, or combinations thereof

83. The porous microparticles as claimed in claim 20 wherein the bioactive is a protein, peptide or polypeptide.

84. The porous microparticles as claimed in claim 77 wherein the organic compound is one or more selected from the group comprising: Bendroflumethiazide, Betamethasone base, Betamethasone valerate, Budesonide, Formoterol fumarate, Hydrochlorothiazide, Hydroflumethiazide, Lysozyme, Para-aminosalicylic acid, Sodium cromoglycate, Sulfadiazine, Sulfadimidine, Sulfamerazine, Trypsin, Insulin, Human growth hormone, Somatotropin, Tissue plasminogen activator, Erthyropoietin, Granulocyte colony stimulating factor (G-CSF), Factor VIII, Interferon-α, Interferon-β, IL-2, Calcitonin, Monoclonal antibodies, Therapeutic proteins/peptides/polypeptides, Therapeutic proteins derived from plants, animals, or microorganisms, and recombinant versions of these products, Monoclonal antibodies, Proteins intended for therapeutic use, cytokines, interferons, enzymes, thrombolytics, and other novel proteins, Immunomodulators, Growth factors, cytokines, and monoclonal antibodies intended to mobilize, stimulate, decrease or otherwise alter the production of hematopoietic cells in vivo, magnesium stearate monosaccharides, for example glucose, galactose, fructose and the like; disaccharides, for example trehalose, maltose, lactose, sucrose and the like; trisaccharides, for example raffinose, acarbose, melezitose and the like; cyclic oligosaccharides/cyclodextrins, for example hydroxpropyl-β-cyclodextrin, hydroxyethyl-β-cyclodextrin, α-cyclodextrin, β-cyclodextrin, γ-cyclodextrin, methyl-β-cyclodextrin, dimethyl-β-cyclodextrin, sulfobutylether-β-cyclodextrin, randomly methylated-β-cyclodextrin and the like; soluble polymers, for example polyvinylpyrrolidone such as PVP 10,000, PVP 40,000, PVP 1,300,00, polyethylene glycol and the like; sugar alcohols/polyols, for example mannitol, xylitol, sorbitol and the like; amino sugars and oligosaccharides, for example inulin and maltodextrin and the like; polysaccharides, for example starch, glycogen and the like; cellulose and cellulose derivatives, for example methylcellulose, ethylcellulose, hydroxypropylmethyl cellulose and the like; deoxy, amino and other sugar derivatives, for example deoxy-glucose, deoxy-ribose, galactosamine and the like, alpha and beta adrenoreceptor agonists for example salbutamol, salmeterol, terbutaline, bambuterol, clenbuterol, metaproterenol, fenoterol, rimiterol, reproterol, bitolterol, tulobuterol, isoprenaline, isoproterenol and the like and their salts, anticholinergics for example ipratropium, oxitropium and tiotropium and the like and their salts, glucocorticoids for example beclomethasone, betamethasone, budesonide, ciclesonide, formoterol, fluticasone, mometasone, triamcinolone and the like and their salts and esters, antiallergics for example nedocromil sodium and sodium cromoglycate and the like, leukotriene inhibitors and antagonists for example montelukast, pranlukast, zafirlukast and zileuton and the like, xanthines for example aminophylline, diprophylline, etofylline, proxyphylline, theobromine and theophylline and the like, anti-infectives for example tobramycin, amikacin, ciprofloxacin, gentamicin, para-aminosalicylic acid, rifampicin, isoniazid, capreomycin, acyclovir and ritonavir and the like, antihistamines for example, terfenadine, cetrizine, loratadine and the like, pain control substances for example morphine and codeine and the like and their salts, and combinations thereof.

85. A pharmaceutical composition comprising substantially pure organic bioactive porous microparticles, the composition may comprise a pharmaceutically acceptable excipient or adjuvant, the pharmaceutical composition may be in the form of a powder.
